(19) United States
(12) Patent Application Publication
(10) Pub. No.: US 2017/0207398 A1
(43) Pub. Date: Jul. 20, 2017

(54) ELECTRON TRANSPORT MATERIAL AND ORGANIC ELECTROLUMINESCENT DEVICE COMPRISING THE SAME

(71) Applicant: Rohm and Haas Electronic Materials Korea Ltd., Cheonan (KR)

(72) Inventors: Jeong-Eun Yang, Suwon (US); Su-Hyun Lee, Suwon (KR); Chi-Sik Kim, Hwaseong (KR); Young-Jun Cho, Seongnam (KR); Kyung-Hoon Choi, Hwaseong (KR); Sang-Hee Cho, Suwon (KR); Jeong-Hwan Jeon, Gwangju-si (KR); Hong-Yeop Na, Seoul (KR)

(21) Appl. No.: 15/326,628

(22) PCT Filed: Jul. 16, 2015
(86) PCT No.: PCT/KR2015/007403

§ 371 Date: Jan. 16, 2017

(30) Foreign Application Priority Data
Jul. 17, 2014 (KR) 10-2014-0090382
Jul. 14, 2015 (KR) 10-2015-0099686

(51) Int. Cl. H01L 51/00; C07D 495/04; H01L 51/50; C07D 405/14; C09K 11/02; C07D 491/048; C09K 11/06

(52) U.S. Cl. CPC H01L 51/0071; C07D 491/048; H01L 51/0067; C07D 495/04; H01L 51/0061; H01L 51/0072; H01L 51/0058; H01L 51/006; C09K 11/06; H01L 51/0073; H01L 51/0052; H01L 51/0077; C07D 405/14; C09K 11/025; H01L 51/5004; C09K 2211/1011; C09K 2211/1007; C09K 2211/1014; H01L 51/5076; H01L 2251/552

(57) ABSTRACT

The present invention relates to an electron transport material comprising a compound of a certain structure, and an organic electroluminescent device comprising the same. The organic electroluminescent device comprising the electron transport material of the present invention has a low driving voltage, high luminous efficiency and good color purity, and thus effectively shows blue emission.

### TECHNICAL FIELD

The present invention relates to an electron transport material and an organic electroluminescent device comprising the same.

### BACKGROUND ART

An electroluminescent (EL) device is a self-light-emitting device with the advantages of providing a wider viewing angle, a greater contrast ratio, and a faster response time. The first organic EL device was developed by Eastman Kodak, by using small aromatic diamine molecules and aluminum complexes as materials for forming a light-emitting layer (see Appl. Phys. Lett. 51, 913, 1987).

An organic EL device changes electric energy into light by the application of electric power into an organic light-emitting material, and commonly comprises an anode, a cathode, and an organic layer formed between the two electrodes. The organic layer of the organic EL device may be composed of a hole injection layer (HIL), a hole transport layer (HTL), an electron blocking layer (EBL), a light-emitting layer (EML) (containing host and dopant materials), an electron buffer layer, a hole blocking layer (HBL), an electron transport layer (ETL), an electron injection layer (EIL), etc.; the materials used in the organic layer can be classified into a hole injection material, a hole transport material, an electron blocking material, a light-emitting material, an electron buffer material, a hole blocking material, an electron transport material, an electron injection material, etc., depending on functions. In the organic EL device, holes from an anode and electrons from a cathode are injected into a light-emitting layer by the application of electric voltage, and an exciton having high energy is produced by the recombination of holes and electrons. The organic light-emitting compound moves into an excited state by the energy and emits light from energy when the organic light-emitting compound returns to the ground state from the excited state.

The most important factor determining luminous efficiency in an organic EL device is light-emitting materials. The light-emitting materials are required to have the following features: high quantum efficiency, high movement degree of an electron and a hole, and formability of a uniform and stable layer. The light-emitting materials are classified into blue light-emitting materials, green light-emitting materials, and red light-emitting materials according to the light-emitting color, and further include yellow light-emitting materials or orange light-emitting materials. Furthermore, the light-emitting material is classified into a host material and a dopant material in a functional aspect. Recently, an urgent task is the development of an organic EL device having high efficacy and long lifespan. In particular, the development of highly excellent light-emitting material compared to conventional light-emitting materials is urgently required considering the EL properties necessary for medium- and large-sized OLED panels. For this, preferably, as a solvent in a solid state and an energy transmitter, a host material should have high purity and a suitable molecular weight in order to be deposited under vacuum. Furthermore, a host material is required to have high glass transition temperature and pyrolysis temperature for guaranteeing thermal stability, high electrochemical stability for long lifespan, easy formability of an amorphous thin film, good adhesion with adjacent layers, and no movement between layers.

Meanwhile, in an organic EL device, an electron transport material actively transports electrons from a cathode to a light-emitting layer and inhibits transport of holes which are not recombined in the light-emitting layer to increase recombination opportunity of holes and electrons in the light-emitting layer. Thus, electron-affinitive materials are used as an electron transport material. Organic metal complexes having light-emitting function such as Alq₃ are excellent in transporting electrons, and thus have been conventionally used as an electron transport material. However, Alq₃ has problems in that it moves to other layers and shows reduction of color purity when used in blue light-emitting devices. Therefore, new electron transport materials have been required, which do not have the above problems, are highly electron-affinitive, and quickly transport electrons in organic EL devices to provide organic EL devices having high luminous efficiency.

Korean Patent Application Laying-open No. 1 0-201 0-01 05099 discloses compounds having a carbazole backbone fused with benzofuran or benzothiophene wherein a nitrogen-containing heterocyclic group is bonded to the nitrogen atom of the carbazole. However, the above literature does not specifically disclose an organic EL device using the above compounds as an electron transport material.

### DISCLOSURE OF THE INVENTION

### Problems to be Solved

The object of the present invention is to provide an electron transport material which can prepare an organic EL device having high efficiency.

### Solution to Problems

The above objective can be achieved by an electron transport material comprising the compound represented by the following formula 1:

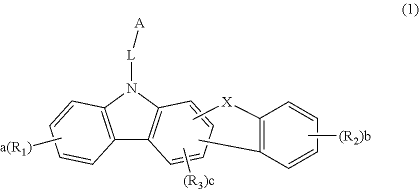

Wherein

A represents a substituted or unsubstituted 5- to 30-membered heteroaryl group;

L represents a single bond, a substituted or unsubstituted (C6-C30)arylene group, or a substituted or unsubstituted 5- to 30-membered heteroarylene group;

X represents O or S;

R₁ and R₂ each independently represent a hydrogen, deuterium, a halogen, a cyano group, a substituted or unsubstituted (C1-C30)alkyl group, a substituted or unsubstituted (C6-C30)aryl group, a substituted or unsubstituted 5- to 30-membered heteroaryl group, a substituted or unsubstituted (C6-C30)aryl(C1-C30)alkyl group, a substituted or unsubstituted (C3-C30)cycloalkyl group, a substituted or unsubstituted (C1-C30)alkoxy group, a substituted or unsubstituted tri(C1-C30)alkylsilyl group, a substituted or unsubstituted tri(C6-C30)arylsilyl group, a substituted or unsubstituted di(C1-C30)alkyl(C6-C30)arylsilyl group, a substituted or unsubstituted (C1-C30)alkyldi(C6-C30)arylsilyl group, a substituted or unsubstituted mono- or di-(C1-C30)alkylamino group, a substituted or unsubstituted mono- or di-(C6-C30)arylamino group, or a substituted or unsubstituted (C1-C30)alkyl(C6-C30)arylamino group; or may be linked to an adjacent substituent(s) to form a substituted or unsubstituted mono- or polycyclic, (C3-C30) alicyclic or aromatic ring whose carbon atom(s) may be replaced with at least one hetero atom selected from nitrogen, oxygen, and sulfur;

R₃ represents a hydrogen, deuterium, a halogen, a cyano group, a substituted or unsubstituted (C1-C30)alkyl group, a substituted or unsubstituted (C6-C30)aryl group, or a substituted or unsubstituted 5- to 30-membered heteroaryl group; or may be linked to an adjacent substituent(s) to form a substituted or unsubstituted mono- or polycyclic, (C3-C30) alicyclic or aromatic ring whose carbon atom(s) may be replaced with at least one hetero atom selected from nitrogen, oxygen, and sulfur;

a and b each independently represent an integer of 1 to 4; where a or b is an integer of 2 or more, each of R₁ or each of R₂ may be the same or different;

c represents an integer of 1 to 2; where c is an integer of 2, each of R₃ may be the same or different; and

the heteroaryl(ene) group contains at least one hetero atom selected from B, N, O, S, Si, and P.

### Effects of the Invention

When using an electron transport material according to the present invention, an organic EL device with high efficiency is provided and the production of a display device or or a lighting device is possible by using the organic EL device.

### BRIEF DESCRIPTION OF THE DRAWINGS

FIG. 1 shows the typical cross-sectional diagram of an organic EL device comprising an electron transport layer comprising an electron transport material according to one embodiment of the present invention.

FIG. 2 briefly shows the relationship of energy gap between the layers disposed in an organic EL device according to one embodiment of the present invention.

FIG. 3 shows graphs of current efficiency vs. luminance of organic EL devices according to one embodiment of the present invention and the conventional technique.

### EMBODIMENTS OF THE INVENTION

Hereinafter, the present invention will be described in detail. However, the following description is intended to explain the invention, and is not meant in any way to restrict the scope of the invention.

The compound represented by formula 1 will be described in detail as follows. Herein, “(C1-C30)alkyl” is meant to be a linear or branched alkyl(ene) chain having 1 to 30 carbon atoms constituting the chain, in which the number of carbon atoms is preferably 1 to 10, more preferably 1 to 6, and includes methyl, ethyl, n-propyl, isopropyl, n-butyl, isobutyl, tert-butyl, etc. “(C2-C30)alkenyl” is meant to be a linear or branched alkenyl chain having 2 to 30 carbon atoms constituting the chain, in which the number of carbon atoms is preferably 2 to 20, more preferably 2 to 10, and includes vinyl, 1-propenyl, 2-propenyl, 1-butenyl, 2-butenyl, 3-butenyl, 2-methylbut-2-enyl, etc. “(C2-C30)alkynyl” is a linear or branched alkynyl chain having 2 to 30 carbon atoms constituting the chain, in which the number of carbon atoms is preferably 2 to 20, more preferably 2 to 10, and includes ethynyl, 1-propynyl, 2-propynyl, 1-butynyl, 2-butynyl, 3-butynyl, 1-methylpent-2-ynyl, etc. “(C3-C30)cycloalkyl” is a mono- or polycyclic hydrocarbon having 3 to 30 carbon atoms in a ring backbone, in which the number of carbon atoms is preferably 3 to 20, more preferably 3 to 7, and includes cyclopropyl, cyclobutyl, cyclopentyl, cyclohexyl, etc. “3- to 7-membered heterocycloalkyl” is a cycloalkyl having at least one heteroatom selected from B, N, O, S, Si, and P, preferably O, S, and N, and 3 to 7 ring backbone atoms, and includes tetrahydrofuran, pyrrolidine, thiolan, tetrahydropyran, etc. “(C6-C30)aryl(ene)” is a monocyclic or fused ring derived from an aromatic hydrocarbon having 6 to 30 carbon atoms in a ring backbone, in which the number of carbon atoms in a ring backbone is preferably 6 to 20, more preferably 6 to 15, and includes phenyl, biphenyl, terphenyl, naphthyl, binaphthyl, phenylnaphthyl, naphthylphenyl, fluorenyl, phenylfluorenyl, benzofluorenyl, dibenzofluorenyl, phenanthrenyl, phenylphenanthrenyl, anthracenyl, indenyl, triphenylenyl, pyrenyl, tetracenyl, perylenyl, chrysenyl, naphthacenyl, fluoranthenyl, etc. “5- to 30-membered heteroaryl(ene)” is an aryl group having at least one, preferably 1 to 4 heteroatom selected from the group consisting of B, N, O, S, Si, and P, and 5 to 30 ring backbone atoms; is a monocyclic ring, or a fused ring condensed with at least one benzene ring; may be partially saturated; may be one formed by linking at least one heteroaryl or aryl group to a heteroaryl group via a single bond(s); and includes a monocyclic ring-type heteroaryl including furyl, thiophenyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, etc., and a fused ring-type heteroaryl including benzofuranyl, benzothiophenyl, isobenzofuranyl, dibenzofuranyl, dibenzothiophenyl, benzonaphtothiophenyl, benzimidazolyl, benzothiazolyl, benzisothiazolyl, benzisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenanthridinyl, benzodioxolyl, etc. “Halogen” includes F, Cl, Br, and I.

In one embodiment of the present invention, the compound of formula 1 may be represented by one of the following formulae 2 to 7:

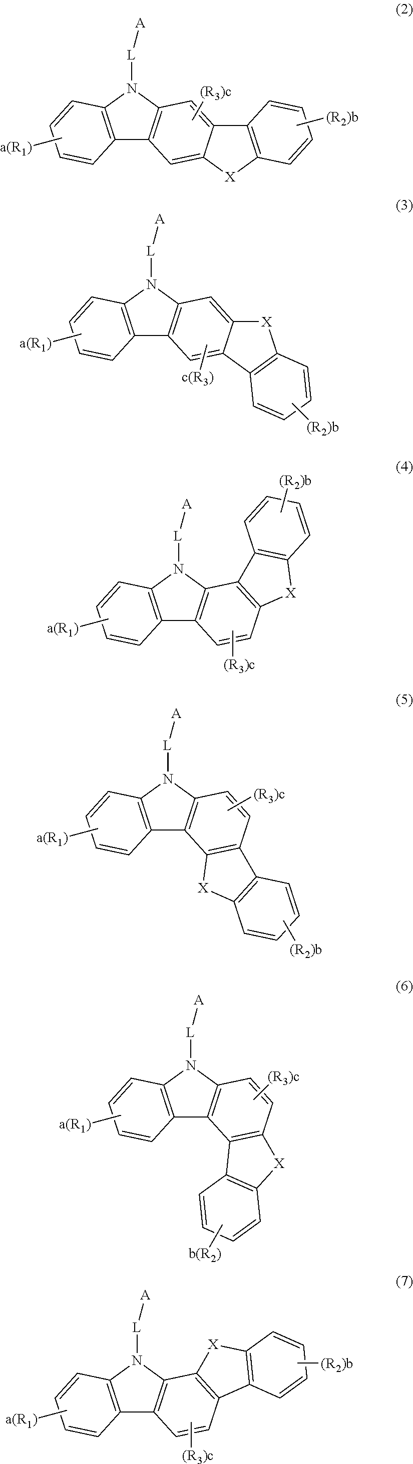

wherein A, L, R₁ to R₃, a, b, and c are as defined in formula 1.

Herein, “substituted” in the expression “substituted or unsubstituted” means that a hydrogen atom in a certain functional group is replaced with another atom or group, i.e., a substituent. Substituents of the substituted alkyl group, the substituted alkoxy, the substituted cycloalkyl group, the substituted aryl(ene) group, the substituted heteroaryl(ene) group, the substituted trialkylsilyl group, the substituted triarylsilyl group, the substituted dialkylarylsilyl group, the substituted alkyldiarylsilyl group, the substituted mono- or di-arylamino group, the substituted mono- or di-alkylamino group, the substituted alkylarylamino group, the substituted aralkyl group, and the substituted mono- or polycyclic, (C3-C30) alicyclic or aromatic ring in A, L, and R₁ to R₃ of the above formulae are each independently at least one selected from the group consisting of deuterium; a halogen; a cyano group; a carboxyl group; a nitro group; a hydroxyl group; a (C1-C30)alkyl group; a halo(C1-C30)alkyl group; a (C2-C30)alkenyl group; a (C2-C30)alkynyl group; a (C1-C30)alkoxy group; a (C1-C30)alkylthio group; a (C3-C30)cycloalkyl group; a (C3-C30)cycloalkenyl group; a 3- to 7-membered heterocycloalkyl group; a (C6-C30)aryloxy group; a (C6-C30)arylthio group; a 3- to 30-membered heteroaryl group which is unsubstituted or substituted with a (C1-C30)alkyl group or a (C6-C30)aryl group; a (C6-C30)aryl group which is unsubstituted or substituted with a (C1-C30)alkyl group or a 3- to 30-membered heteroaryl group; a (C6-C30)aryl group which is substituted with a tri(C1-C30)alkylsilyl group; a (C6-C30)aryl group which is substituted with a tri(C6-C30)arylsilyl group; a tri(C1-C30)alkylsilyl group; a tri(C6-C30)arylsilyl group; a di(C1-C30)alkyl(C6-C30)arylsilyl group; a (C1-C30)alkyldi(C6-C30)arylsilyl group; an amino group; a mono- or di-(C1-C30)alkylamino group; a mono- or di-(C6-C30)arylamino group; a (C1-C30)alkyl(C6-C30)arylamino group; a (C1-C30)alkylcarbonyl group; a (C1-C30)alkoxycarbonyl group; a (C6-C30)arylcarbonyl group; a di(C6-C30)arylboronyl group; a di(C1-C30)alkylboronyl group; a (C1-C30)alkyl(C6-C30)arylboronyl group; a (C6-C30)aryl(C1-C30)alkyl group; and a (C1-C30)alkyl(C6-C30)aryl group; and, preferably, at least one selected from the group consisting of a 5- to 20-membered heteroaryl group; a 5- to 20-membered heteroaryl group which is substituted with a (C1-C20)alkyl group; a 5- to 20-membered heteroaryl group which is substituted with a (C6-C20)aryl group; a (C6-C20)aryl group; a (C6-C20)aryl group which is substituted with a (C1-C20)alkyl group; a (C6-C20)aryl group which is substituted with a 5- to 20-membered heteroaryl group; a (C6-C20)aryl group which is substituted with a tri(C1-C6)alkylsilyl group; a (C6-C20)aryl group which is substituted with a tri(C6-C20)arylsilyl group; and a (C1-C6)alkyl(C6-C20)aryl group.

In formula 1, A represents a substituted or unsubstituted 5- to 30-membered heteroaryl group; preferably, a substituted or unsubstituted 5- to 20-membered heteroaryl group; and more preferably, an unsubstituted 5- to 20-membered heteroaryl group, a 5- to 20-membered heteroaryl group substituted with a 5- to 20-membered heteroaryl group which is unsubstituted or substituted with a (C1-C20)alkyl group or a (C6-C20)aryl group, a 5- to 20-membered heteroaryl group substituted with a (C6-C20)aryl group, a 5- to 20-membered heteroaryl group substituted with a (C6-C20)aryl group substituted with a 5- to 20-membered heteroaryl group, a 5- to 20-membered heteroaryl group substituted with a (C6-C20)aryl group substituted with a tri(C1-C6)alkylsilyl group, a 5- to 20-membered heteroaryl group substituted with a (C6-C20)aryl group substituted with a tri(C6-C20)arylsilyl group, or a 5- to 20-membered heteroaryl group substituted with a (C1-C6)alkyl(C6-C20)aryl group.

The 5- to 30-membered heteroaryl group in the definition of A is preferably a nitrogen-containing heteroaryl group. Specifically, A may represent a substituted or unsubstituted pyridine, a substituted or unsubstituted pyrimidine, a substituted or unsubstituted triazine, a substituted or unsubstituted pyrazine, a substituted or unsubstituted quinoline, a substituted or unsubstituted quinazoline, a substituted or unsubstituted quinoxaline, a substituted or unsubstituted naphthyridine, or a substituted or unsubstituted phenanthroline. More specifically, substituents of the substituted heteroaryl group in the definition of A may represent phenyl; biphenyl; terphenyl; naphthyl; phenanthrenyl; triphenylsilyl; a phenyl, biphenyl, or naphthyl group substituted with a triphenylsilyl group; a fluorenyl group which is unsubstituted or substituted with a (C1-C4)alkyl group or a phenyl group; a phenyl, biphenyl, or naphthyl group substituted with a fluorenyl group which is unsubstituted or substituted with a (C1-C4)alkyl group or a phenyl group; a dibenzothiophenyl group which is unsubstituted or substituted with a (C1-C4)alkyl group; a phenyl, biphenyl, or naphthyl group substituted with a dibenzothiophenyl group which is unsubstituted or substituted with a (C1-C4)alkyl group; a dibenzofuranyl group which is unsubstituted or substituted with a (C1-C4)alkyl group; a phenyl, biphenyl, or naphthyl group substituted with a dibenzofuranyl group which is unsubstituted or substituted with a (C1-C4)alkyl group; a carbazole group which is unsubstituted or substituted with a phenyl group; a phenyl, biphenyl, or naphthyl group substituted with a carbazole group; a benzothiazole group which is unsubstituted or substituted with a (C1-C4)alkyl group; or a phenyl, biphenyl, or naphthyl group substituted with a benzothiazole group which is unsubstituted or substituted with a (C1-C4)alkyl group.

L in formula 1 represents a single bond, a substituted or unsubstituted (C6-C30)arylene group, or a substituted or unsubstituted 5- to 30-membered heteroarylene group; preferably, a single bond, a substituted or unsubstituted (C6-C20)arylene group, or a substituted or unsubstituted 5- to 20-membered heteroarylene group; and more preferably, a single bond, an unsubstituted (C6-C20)arylene group, or an unsubstituted 5- to 20-membered heteroarylene group; and still more preferably, a single bond or an unsubstituted (C6-C12)arylene group. Specifically, L may represent a single bond, phenylene, biphenylene, or naphthylene.

X in formula 1 represents O or S.

R₁ and R₂ in formula 1 each independently represent a hydrogen, deuterium, a halogen, a cyano group, a substituted or unsubstituted (C1-C30)alkyl group, a substituted or unsubstituted (C6-C30)aryl group, a substituted or unsubstituted 5- to 30-membered heteroaryl group, a substituted or unsubstituted (C6-C30)aryl(C1-C30)alkyl group, a substituted or unsubstituted (C3-C30)cycloalkyl group, a substituted or unsubstituted (C1-C30)alkoxy group, a substituted or unsubstituted (C1-C30)alkylsilyl group, a substituted or unsubstituted (C6-C30)arylsilyl group, a substituted or unsubstituted (C6-C30)aryl(C1-C30)alkylsilyl group, a substituted or unsubstituted (C1-C30)alkylamino group, a substituted or unsubstituted (C6-C30)arylamino group, or a substituted or unsubstituted (C1-C30)alkyl(C6-C30)arylamino group; or may be linked to an adjacent substituent(s) to form a substituted or unsubstituted mono- or polycyclic, (C3-C30) alicyclic or aromatic ring whose carbon atom(s) may be replaced with at least one hetero atom selected from nitrogen, oxygen, and sulfur; preferably, a hydrogen, a substituted or unsubstituted (C6-C20)aryl group, or a substituted or unsubstituted 5- to 20-membered heteroaryl group; and more preferably, a hydrogen, a (C6-C20)aryl group which is unsubstituted or substituted with a (C6-C12)aryl group, or a 5- to 20-membered heteroaryl group which is unsubstituted or substituted with a (C6-C20)aryl group. Specifically, R₁ and R₂ may each independently represent a hydrogen, a phenyl group which is unsubstituted or substituted with a (C1-C4)alkyl group; a biphenyl group which is unsubstituted or substituted with a (C1-C4)alkyl group; a terphenyl group which is unsubstituted or substituted with a (C1-C4)alkyl group; a naphthyl group which is unsubstituted or substituted with a (C1-C4)alkyl group; a phenanthrenyl group which is unsubstituted or substituted with a (C1-C4)alkyl group; a fluorenyl group which is unsubstituted or substituted with a (C1-C4)alkyl group; a carbazole group which is unsubstituted or substituted with a (C1-C4)alkyl group or a phenyl group; a dibenzothiophenyl group which is unsubstituted or substituted with a (C1-C4)alkyl group or a phenyl group; or a dibenzofuranyl group which is unsubstituted or substituted with a (C1-C4)alkyl group or a phenyl group.

R₃ in formula 1 represents a hydrogen, deuterium, a halogen, a cyano group, a substituted or unsubstituted (C1-C30)alkyl group, a substituted or unsubstituted (C6-C30)aryl group, or a substituted or unsubstituted 5- to 30-membered heteroaryl group; or may be linked to an adjacent substituent(s) to form a substituted or unsubstituted mono- or polycyclic, (C3-C30) alicyclic or aromatic ring whose carbon atom(s) may be replaced with at least one hetero atom selected from nitrogen, oxygen, and sulfur; and preferably, a hydrogen, a substituted or unsubstituted (C6-C20)aryl group, or a substituted or unsubstituted 5- to 20-membered heteroaryl group. Specifically, R₃ represents a hydrogen.

a and b in formula 1 each independently represent an integer of 1 to 4; preferably, an integer of 1 to 2; where a or b is an integer of 2 or more, each of R₁ or each of R₂ may be the same or different.

c in formula 1 represents an integer of 1 to 2; and preferably, an integer of 1.

The heteroaryl(ene) group in formula 1 contains at least one hetero atom selected from B, N, O, S, Si, and P; and preferably, at least one hetero atom selected from N, O, and S.

According to one embodiment of the present invention, in formula 1, A represents a substituted or unsubstituted 5- to 20-membered heteroaryl group; L represents a single bond, a substituted or unsubstituted (C6-C20)arylene group, or a substituted or unsubstituted 5- to 20-membered heteroarylene group; X represents O or S; R₁ and R₂ each independently represent a hydrogen, a substituted or unsubstituted (C6-C20)aryl group, or a substituted or unsubstituted 5- to 20-membered heteroaryl group; R₃ represents a hydrogen; a and b each independently represent an integer of 1 to 2; and c represents 1.

According to another embodiment of the present invention, in formula 1, A represents an unsubstituted 5- to 20-membered heteroaryl group, a 5- to 20-membered heteroaryl group substituted with a 5- to 20-membered heteroaryl group which is unsubstituted or substituted with a (C1-C20)alkyl group or a (C6-C20)aryl group, a 5- to 20-membered heteroaryl group substituted with a (C6-C20)aryl group, a 5- to 20-membered heteroaryl group substituted with a (C6-C20)aryl group substituted with a 5- to 20-membered heteroaryl group, a 5- to 20-membered heteroaryl group substituted with a (C6-C20)aryl group substituted with a tri(C1-C6)alkylsilyl group, a 5- to 20-membered heteroaryl group substituted with a (C6-C20)aryl group substituted with a tri(C6-C20)arylsilyl group, or a 5- to 20-membered heteroaryl group substituted with a (C1-C6)alkyl(C6-C20)aryl group; L represents a single bond, an unsubstituted (C6-C20)arylene group, or an unsubstituted 5- to 20-membered heteroarylene group; X represents O or S; R₁ and R₂ each independently represent a hydrogen, a (C6-C20)aryl group which is unsubstituted or substituted with a (C6-C12)aryl group, or a 5- to 20-membered heteroaryl group which is unsubstituted or substituted with a (C6-C20)aryl group; R₃ represents a hydrogen; a and b each independently represent an integer of 1 to 2; and c represents an integer of 1.

The compound of formula 1 may be selected from the group consisting of the following compounds, but is not limited thereto:

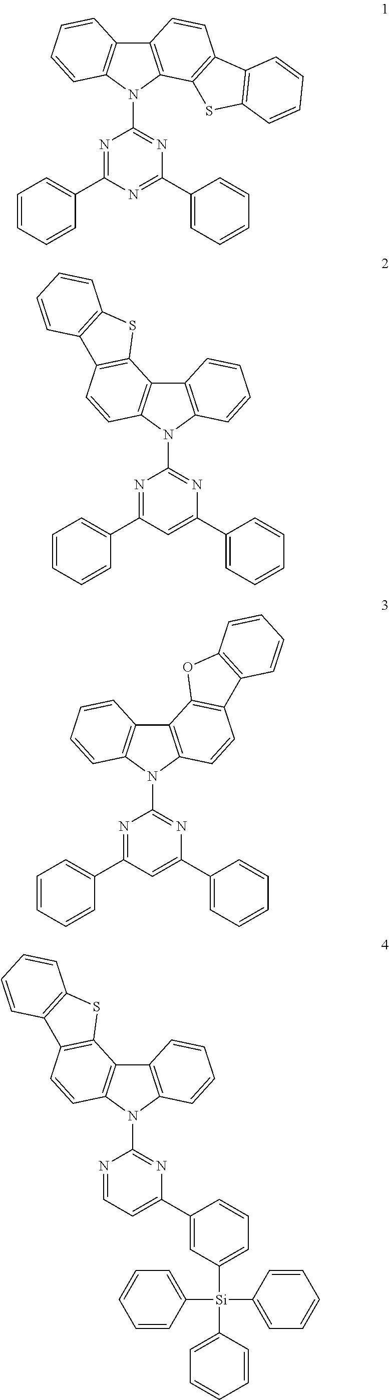

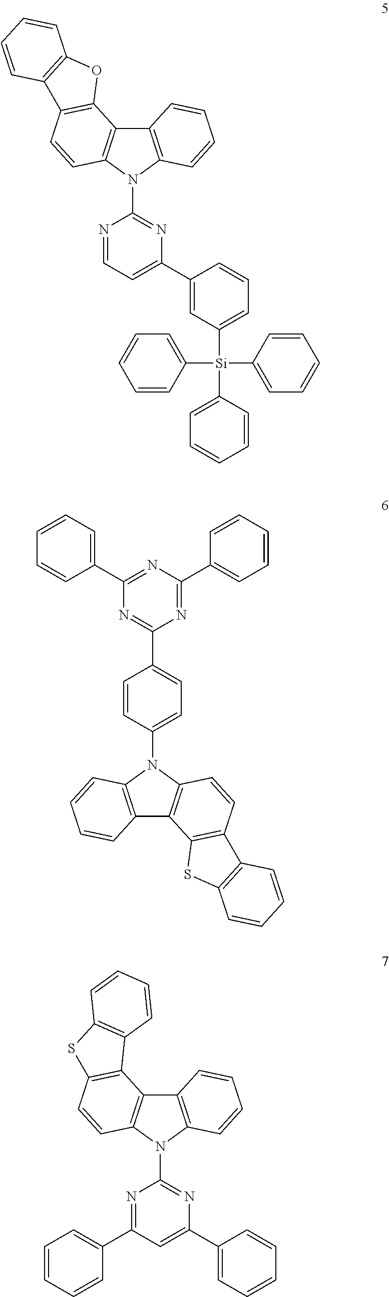 [SMILES: c1ccc([Si](c2ccccc2)(c2ccccc2)c2cccc(-c3ccnc(-n4c5ccccc5c5c6oc7ccccc7c6ccc54)n3)c2)cc1 ; c1ccc(-c2nc(-c3ccccc3)nc(-c3ccc(-n4c5ccccc5c5c6sc7ccccc7c6ccc54)cc3)n2)cc1 ; c1ccc(-c2cc(-c3ccccc3)nc(-n3c4ccccc4c4c5c(ccc43)sc3ccccc35)n2)cc1]

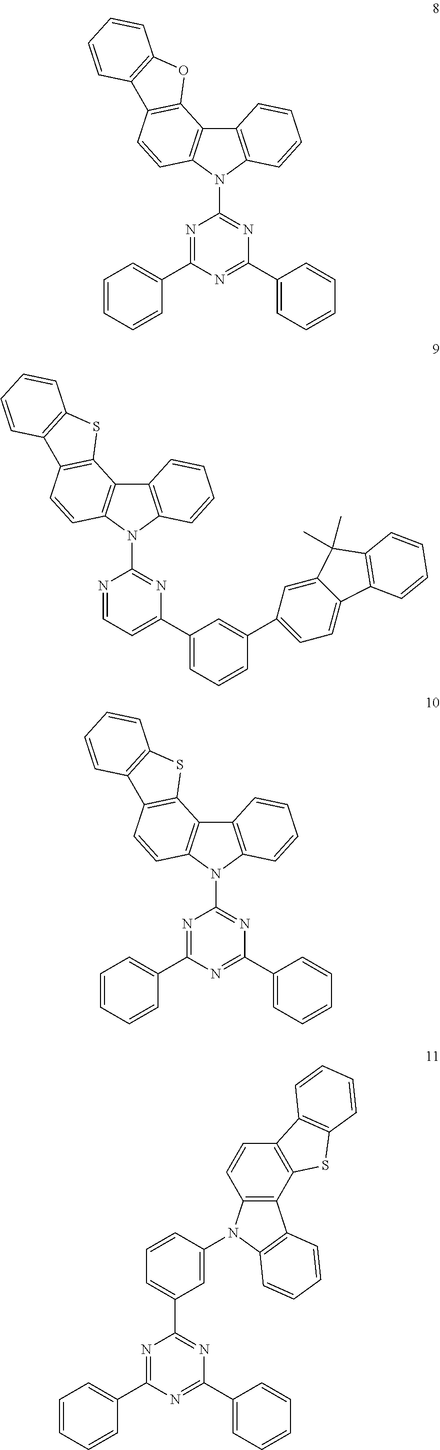

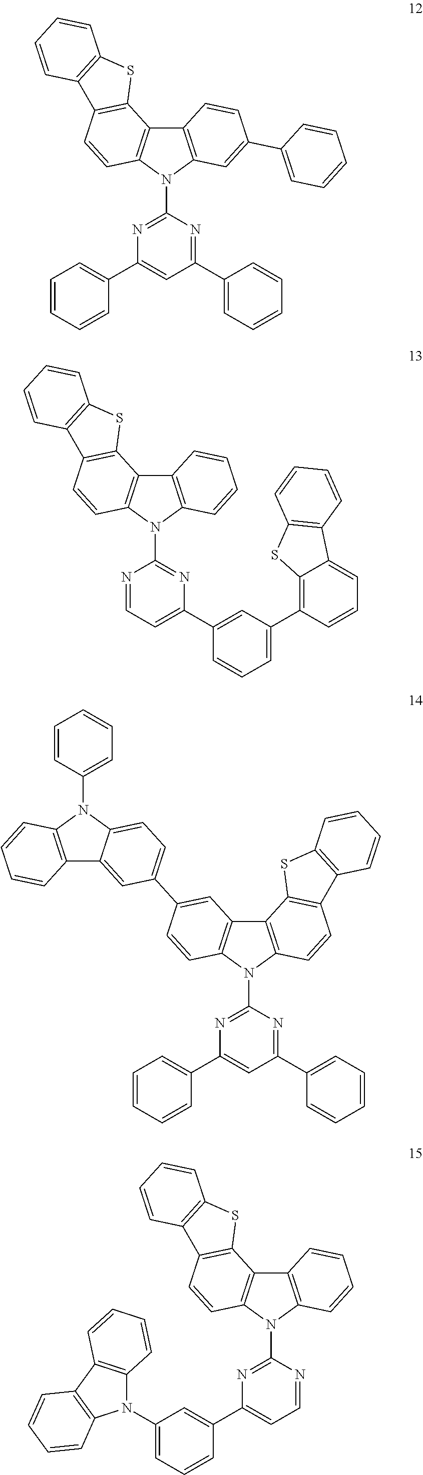

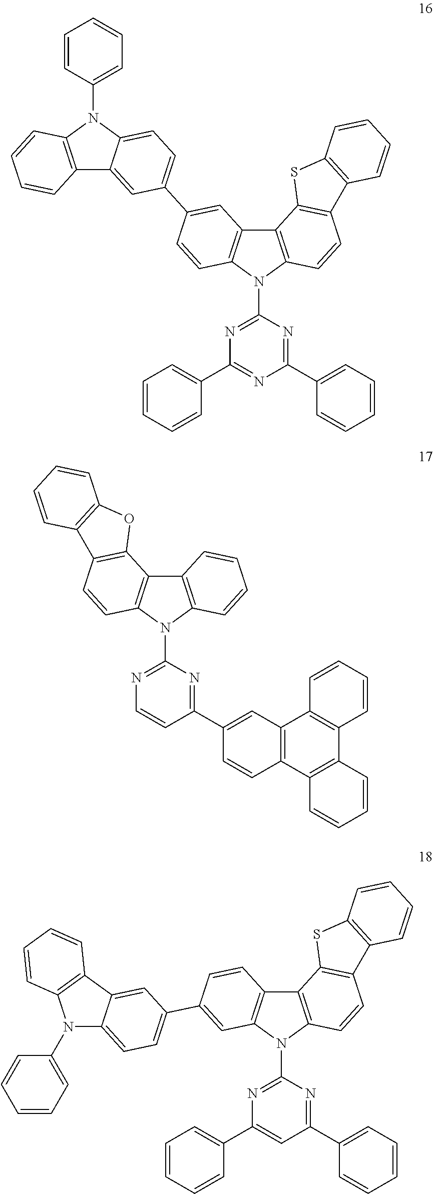 [SMILES: c1ccc(-c2nc(-c3ccccc3)nc(-n3c4ccc(-c5ccc6c(c5)c5ccccc5n6-c5ccccc5)cc4c4c5sc6ccccc6c5ccc43)n2)cc1 ; c1ccc2c(c1)oc1c2ccc2c1c1ccccc1n2-c1nccc(-c2ccc3c4ccccc4c4ccccc4c3c2)n1 ; c1ccc(-c2cc(-c3ccccc3)nc(-n3c4cc(-c5ccc6c(c5)c5ccccc5n6-c5ccccc5)ccc4c4c5sc6ccccc6c5ccc43)n2)cc1]

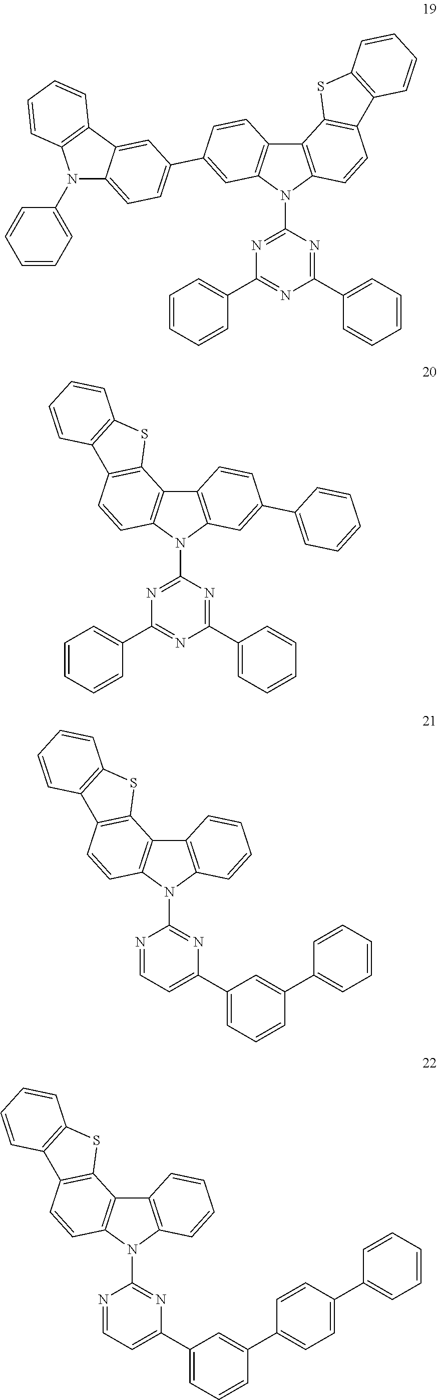

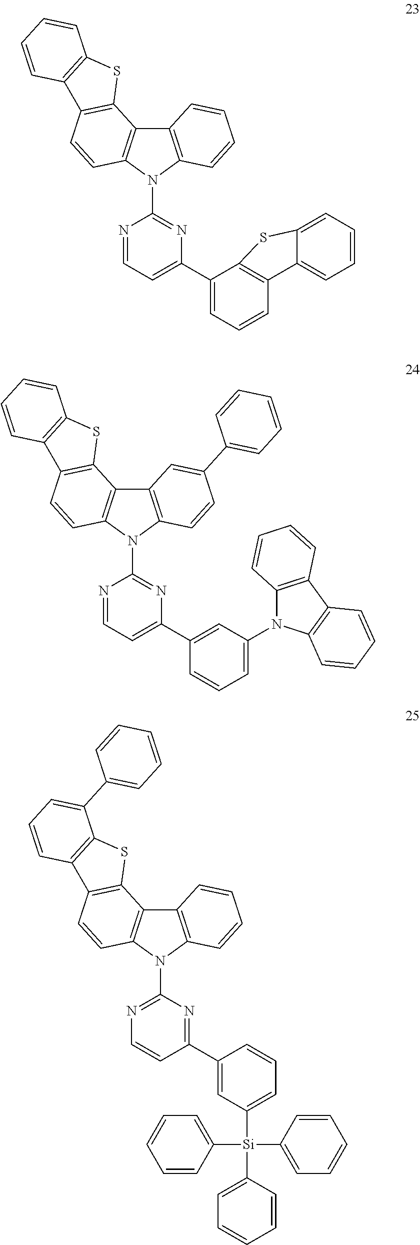 [SMILES: c1ccc2c(c1)sc1c(-c3ccnc(-n4c5ccccc5c5c6sc7ccccc7c6ccc54)n3)cccc12 ; c1ccc(-c2ccc3c(c2)c2c4sc5ccccc5c4ccc2n3-c2nccc(-c3cccc(-n4c5ccccc5c5ccccc54)c3)n2)cc1 ; c1ccc(-c2cccc3c2sc2c3ccc3c2c2ccccc2n3-c2nccc(-c3cccc([Si](c4ccccc4)(c4ccccc4)c4ccccc4)c3)n2)cc1]

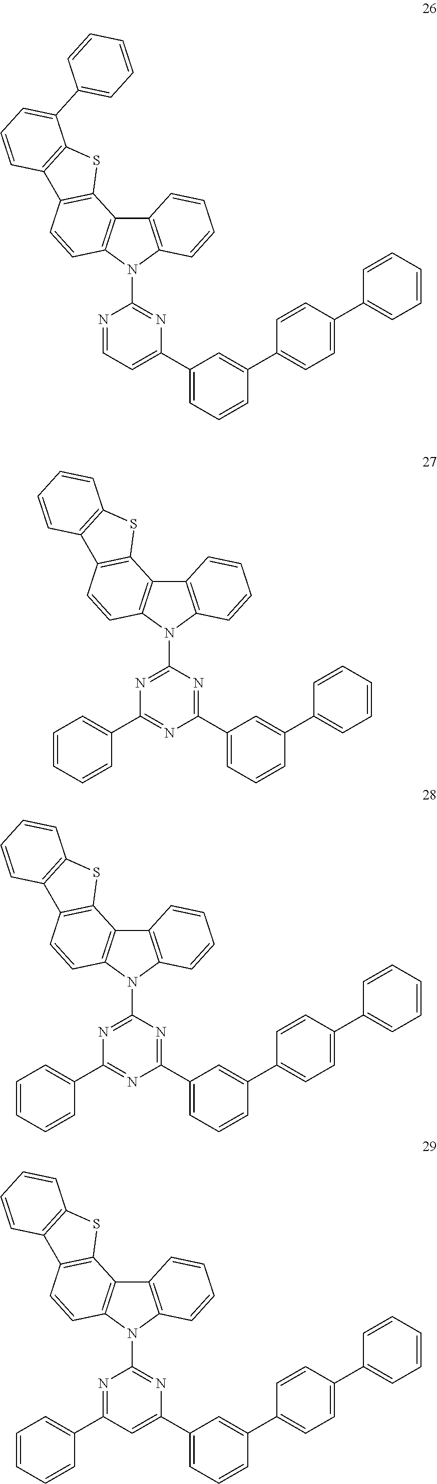

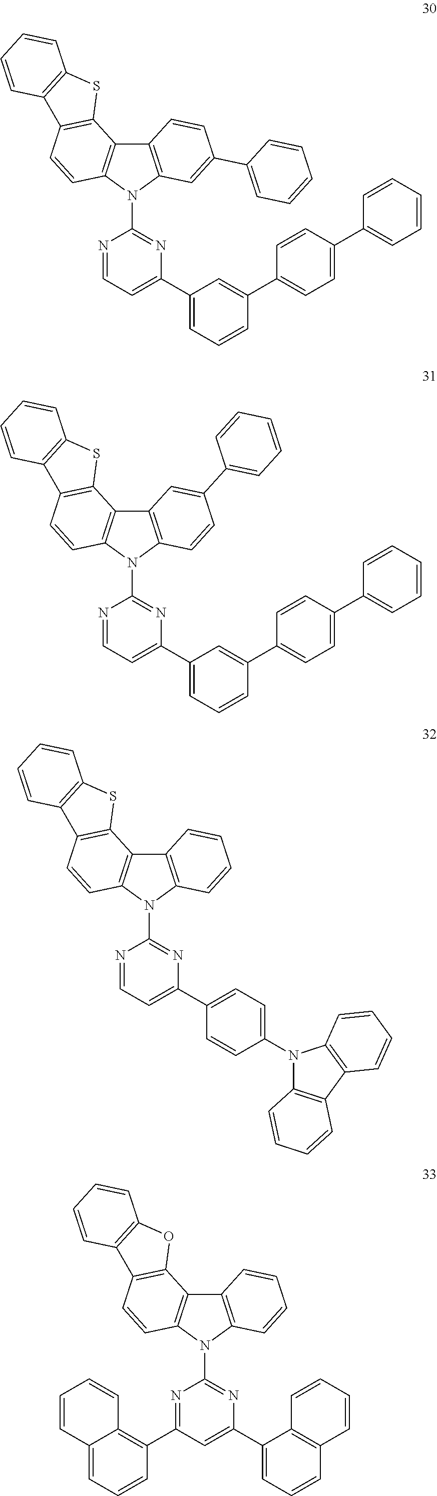 [SMILES: c1ccc(-c2ccc(-c3cccc(-c4ccnc(-n5c6cc(-c7ccccc7)ccc6c6c7sc8ccccc8c7ccc65)n4)c3)cc2)cc1 ; c1ccc(-c2ccc(-c3cccc(-c4ccnc(-n5c6ccc(-c7ccccc7)cc6c6c7sc8ccccc8c7ccc65)n4)c3)cc2)cc1 ; c1ccc2c(c1)sc1c2ccc2c1c1ccccc1n2-c1nccc(-c2ccc(-n3c4ccccc4c4ccccc43)cc2)n1 ; c1ccc2c(-c3cc(-c4cccc5ccccc45)nc(-n4c5ccccc5c5c6oc7ccccc7c6ccc54)n3)cccc2c1]

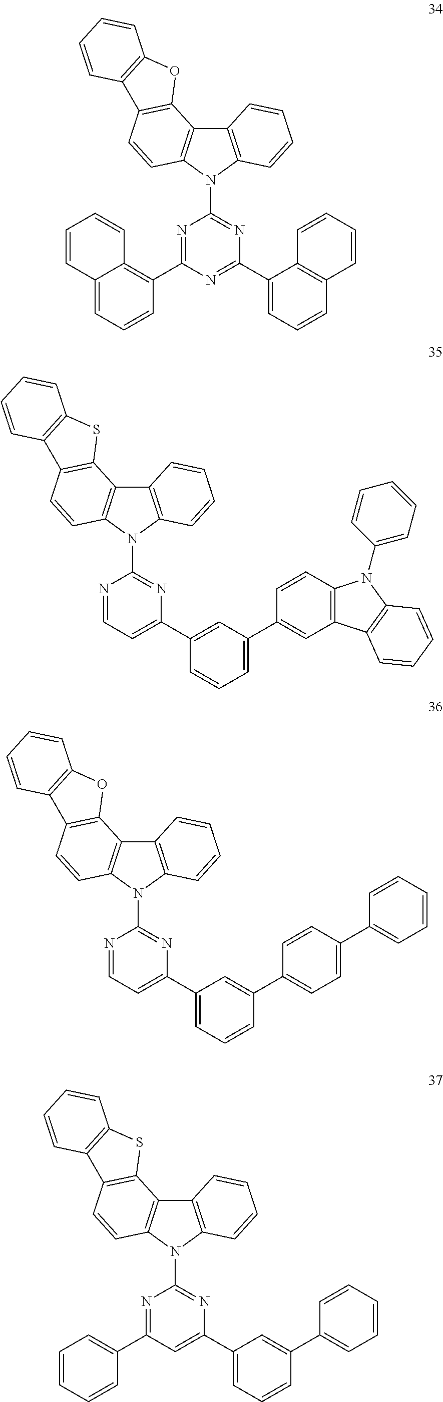 [SMILES: c1ccc2c(-c3nc(-c4cccc5ccccc45)nc(-n4c5ccccc5c5c6oc7ccccc7c6ccc54)n3)cccc2c1 ; c1ccc(-n2c3ccccc3c3cc(-c4cccc(-c5ccnc(-n6c7ccccc7c7c8sc9ccccc9c8ccc76)n5)c4)ccc32)cc1 ; c1ccc(-c2ccc(-c3cccc(-c4ccnc(-n5c6ccccc6c6c7oc8ccccc8c7ccc65)n4)c3)cc2)cc1 ; c1ccc(-c2cccc(-c3cc(-c4ccccc4)nc(-n4c5ccccc5c5c6sc7ccccc7c6ccc54)n3)c2)cc1]

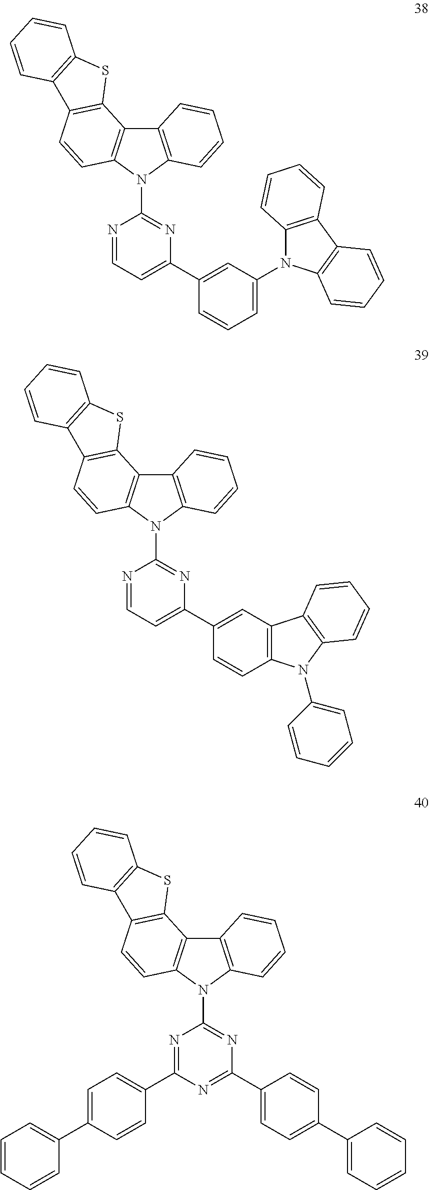 [SMILES: c1cc(-c2ccnc(-n3c4ccccc4c4c5sc6ccccc6c5ccc43)n2)cc(-n2c3ccccc3c3ccccc32)c1 ; c1ccc(-n2c3ccccc3c3cc(-c4ccnc(-n5c6ccccc6c6c7sc8ccccc8c7ccc65)n4)ccc32)cc1 ; c1ccc(-c2ccc(-c3nc(-c4ccc(-c5ccccc5)cc4)nc(-n4c5ccccc5c5c6sc7ccccc7c6ccc54)n3)cc2)cc1]

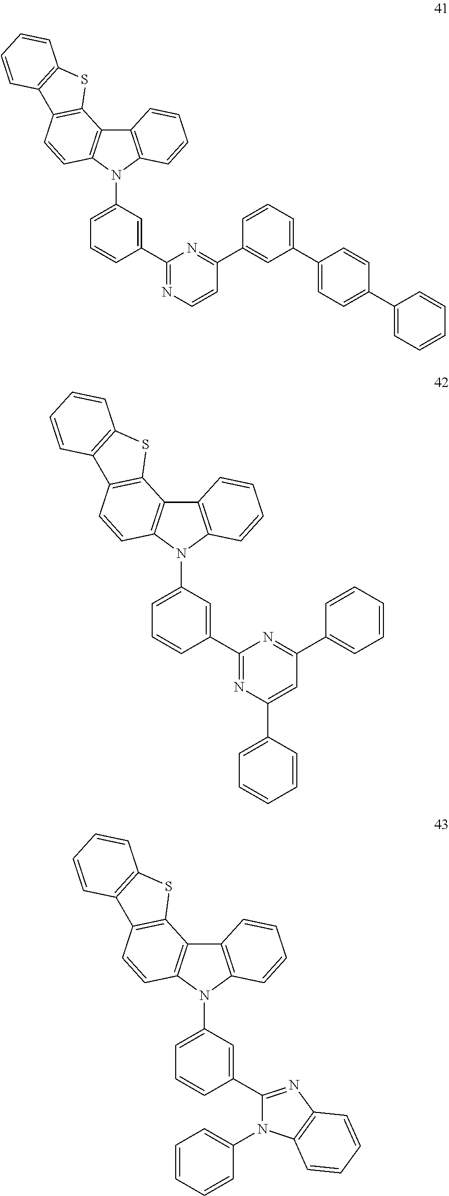 [SMILES: c1ccc(-c2ccc(-c3cccc(-c4ccnc(-c5cccc(-n6c7ccccc7c7c8sc9ccccc9c8ccc76)c5)n4)c3)cc2)cc1 ; c1ccc(-c2cc(-c3ccccc3)nc(-c3cccc(-n4c5ccccc5c5c6sc7ccccc7c6ccc54)c3)n2)cc1 ; c1ccc(-n2c(-c3cccc(-n4c5ccccc5c5c6sc7ccccc7c6ccc54)c3)nc3ccccc32)cc1]

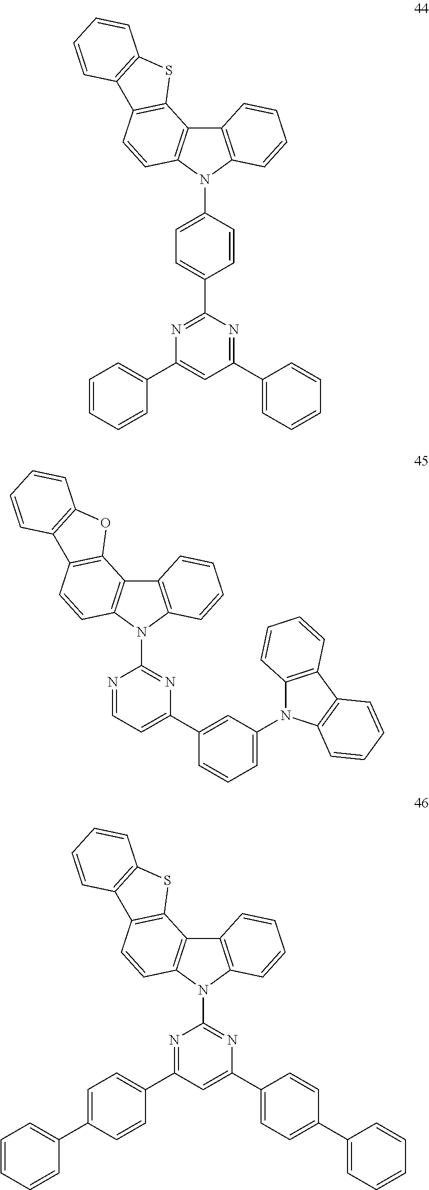 [SMILES: c1ccc(-c2cc(-c3ccccc3)nc(-c3ccc(-n4c5ccccc5c5c6sc7ccccc7c6ccc54)cc3)n2)cc1 ; c1cc(-c2ccnc(-n3c4ccccc4c4c5oc6ccccc6c5ccc43)n2)cc(-n2c3ccccc3c3ccccc32)c1 ; c1ccc(-c2ccc(-c3cc(-c4ccc(-c5ccccc5)cc4)nc(-n4c5ccccc5c5c6sc7ccccc7c6ccc54)n3)cc2)cc1]

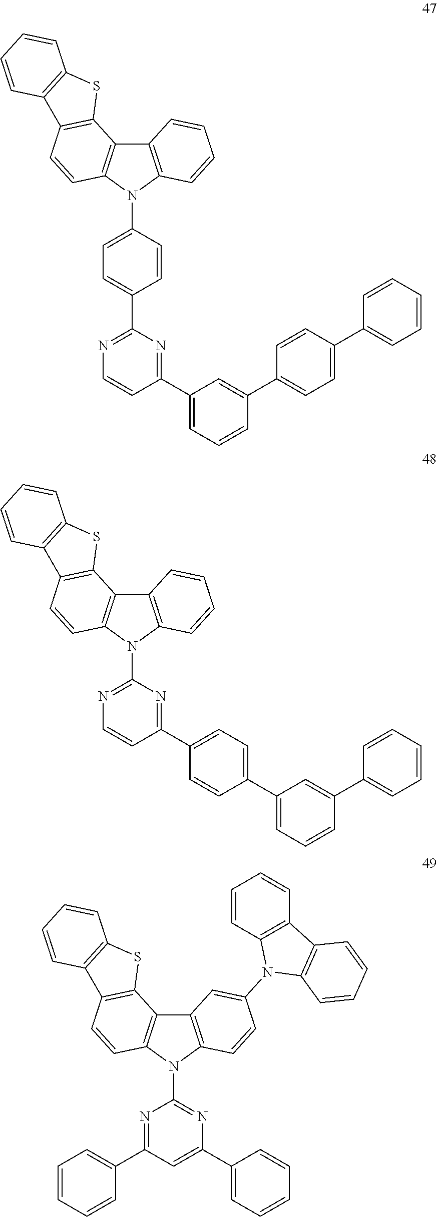

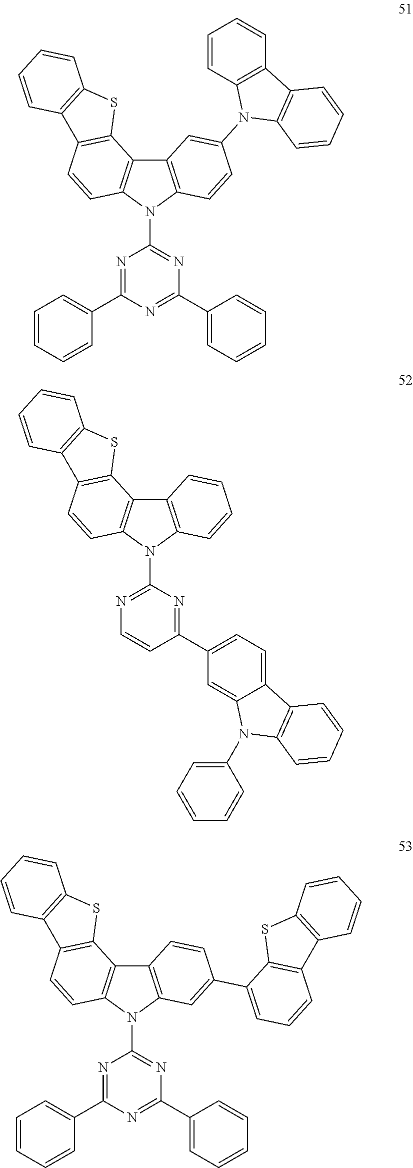

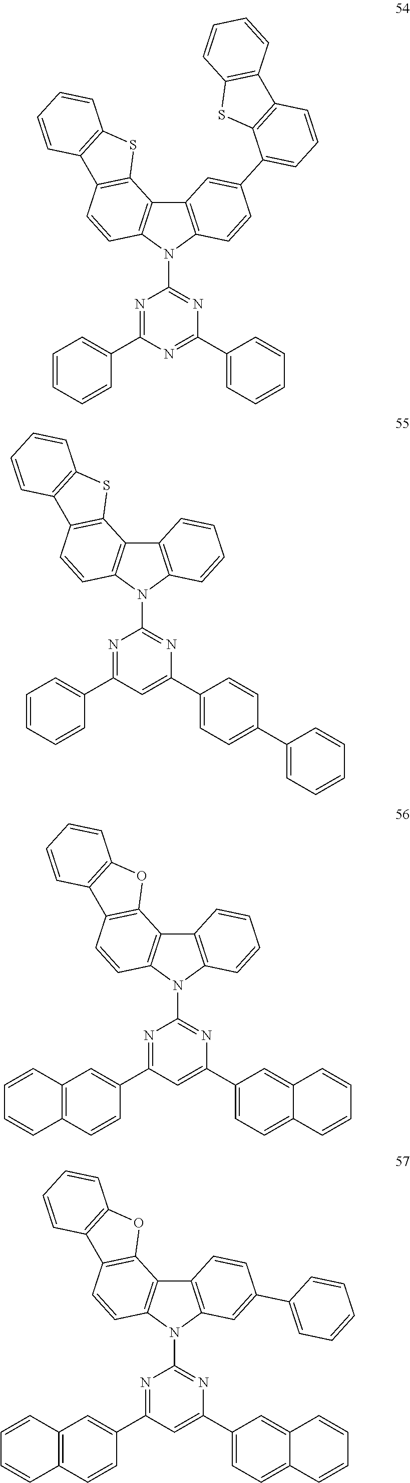

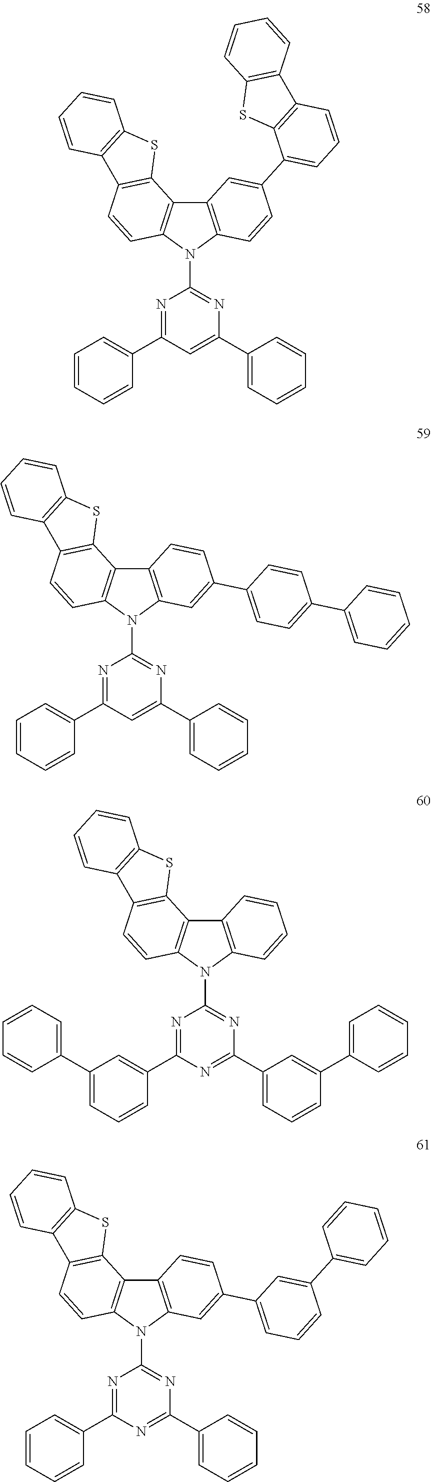 [SMILES: c1ccc(-c2cc(-c3ccccc3)nc(-n3c4ccc(-c5cccc6c5sc5ccccc56)cc4c4c5sc6ccccc6c5ccc43)n2)cc1 ; c1ccc(-c2ccc(-c3ccc4c5c6sc7ccccc7c6ccc5n(-c5nc(-c6ccccc6)cc(-c6ccccc6)n5)c4c3)cc2)cc1 ; c1ccc(-c2cccc(-c3nc(-c4cccc(-c5ccccc5)c4)nc(-n4c5ccccc5c5c6sc7ccccc7c6ccc54)n3)c2)cc1 ; c1ccc(-c2cccc(-c3ccc4c5c6sc7ccccc7c6ccc5n(-c5nc(-c6ccccc6)nc(-c6ccccc6)n5)c4c3)c2)cc1]

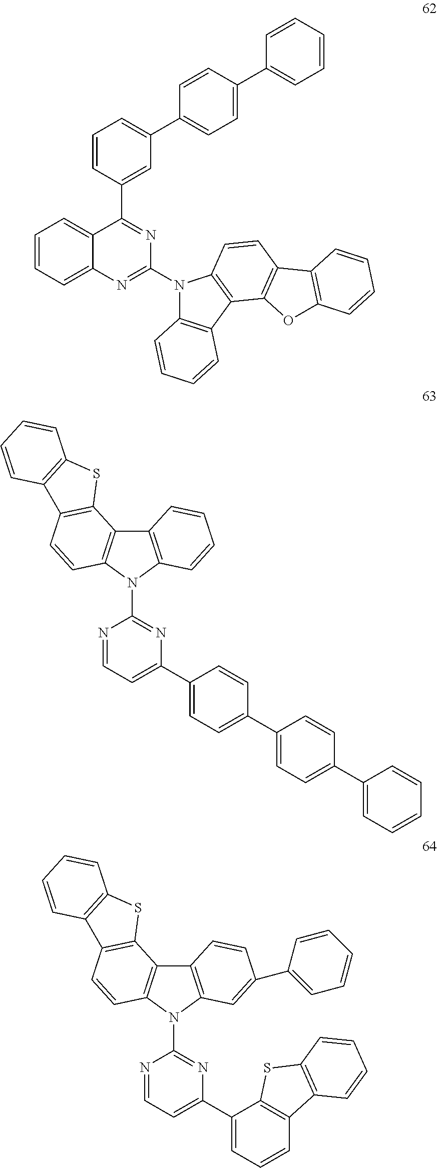 [SMILES: c1ccc(-c2ccc(-c3cccc(-c4nc(-n5c6ccccc6c6c7oc8ccccc8c7ccc65)nc5ccccc45)c3)cc2)cc1 ; c1ccc(-c2ccc(-c3ccc(-c4ccnc(-n5c6ccccc6c6c7sc8ccccc8c7ccc65)n4)cc3)cc2)cc1 ; c1ccc(-c2ccc3c4c5sc6ccccc6c5ccc4n(-c4nccc(-c5cccc6c5sc5ccccc56)n4)c3c2)cc1]

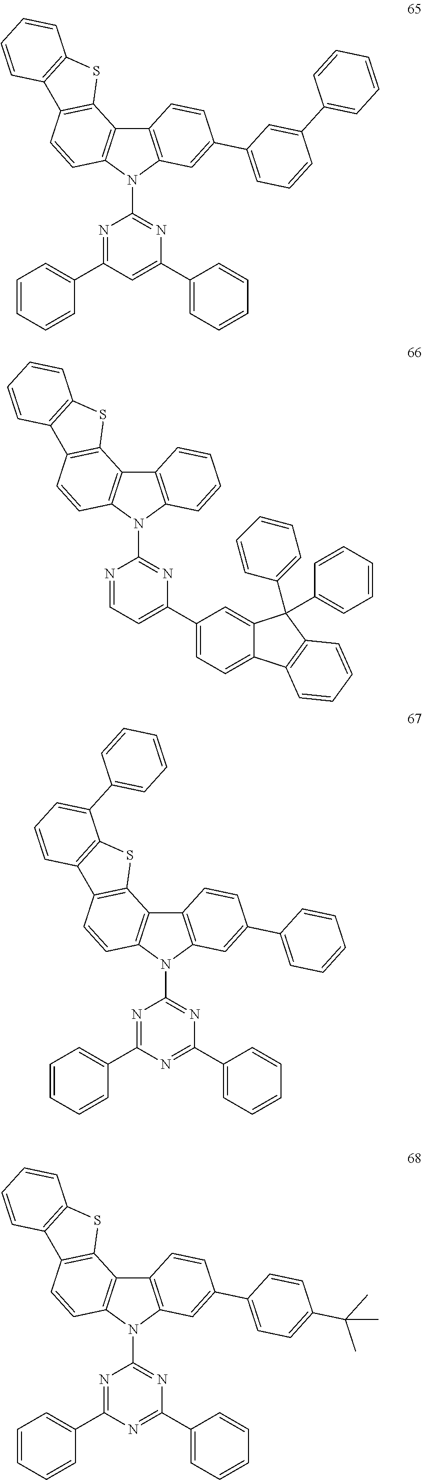

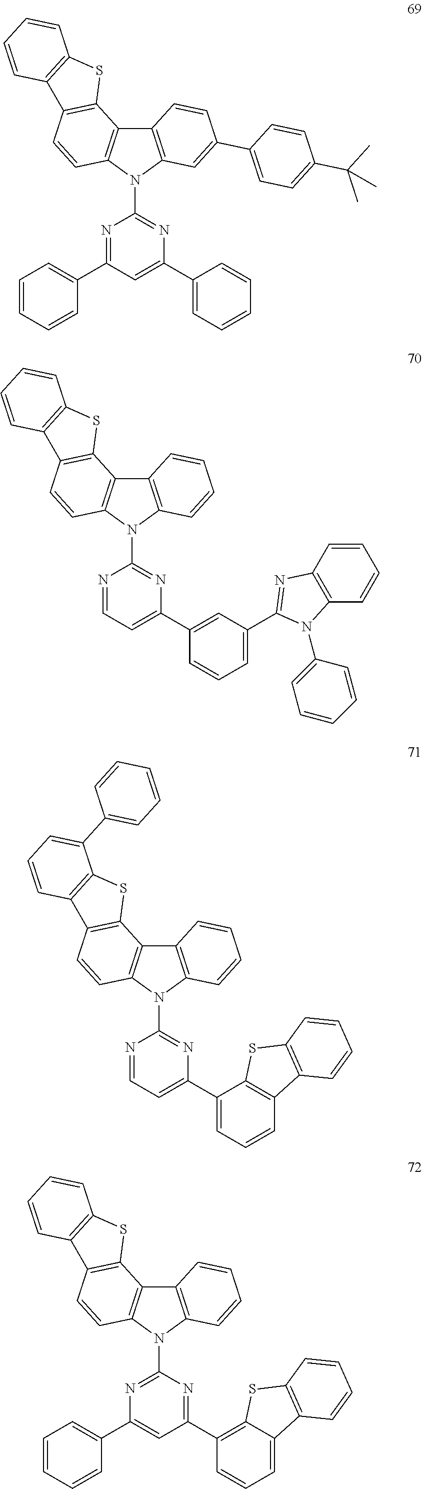

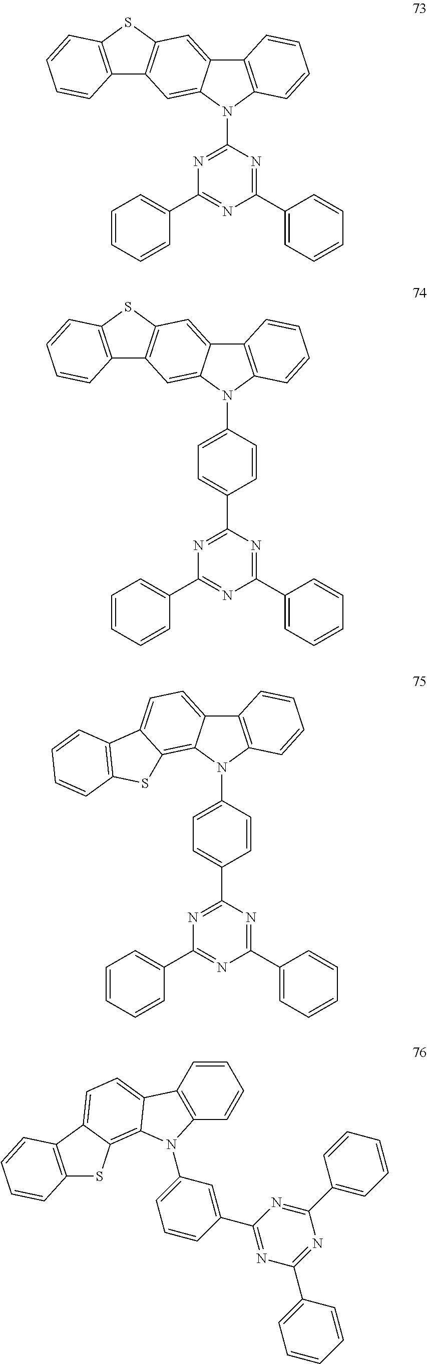 [SMILES: c1ccc(-c2nc(-c3ccccc3)nc(-n3c4ccccc4c4cc5sc6ccccc6c5cc43)n2)cc1 ; c1ccc(-c2nc(-c3ccccc3)nc(-c3ccc(-n4c5ccccc5c5cc6sc7ccccc7c6cc54)cc3)n2)cc1 ; c1ccc(-c2nc(-c3ccccc3)nc(-c3ccc(-n4c5ccccc5c5ccc6c7ccccc7sc6c54)cc3)n2)cc1 ; c1ccc(-c2nc(-c3ccccc3)nc(-c3cccc(-n4c5ccccc5c5ccc6c7ccccc7sc6c54)c3)n2)cc1]

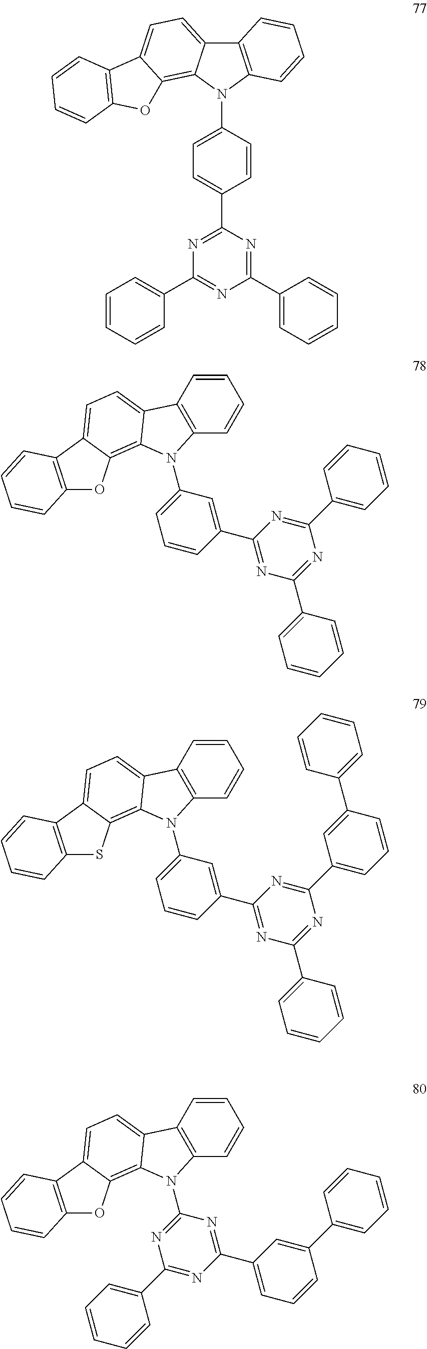

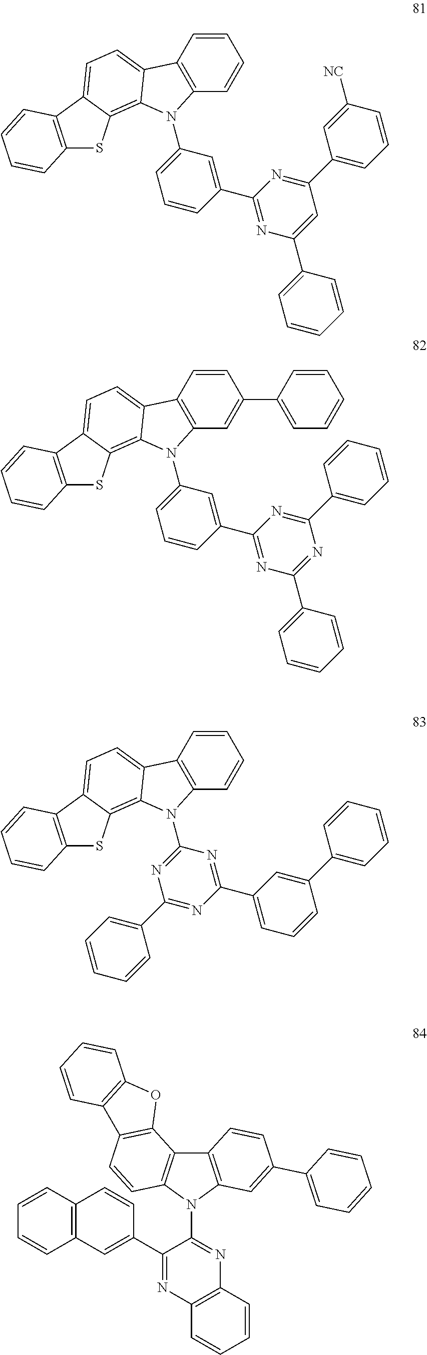

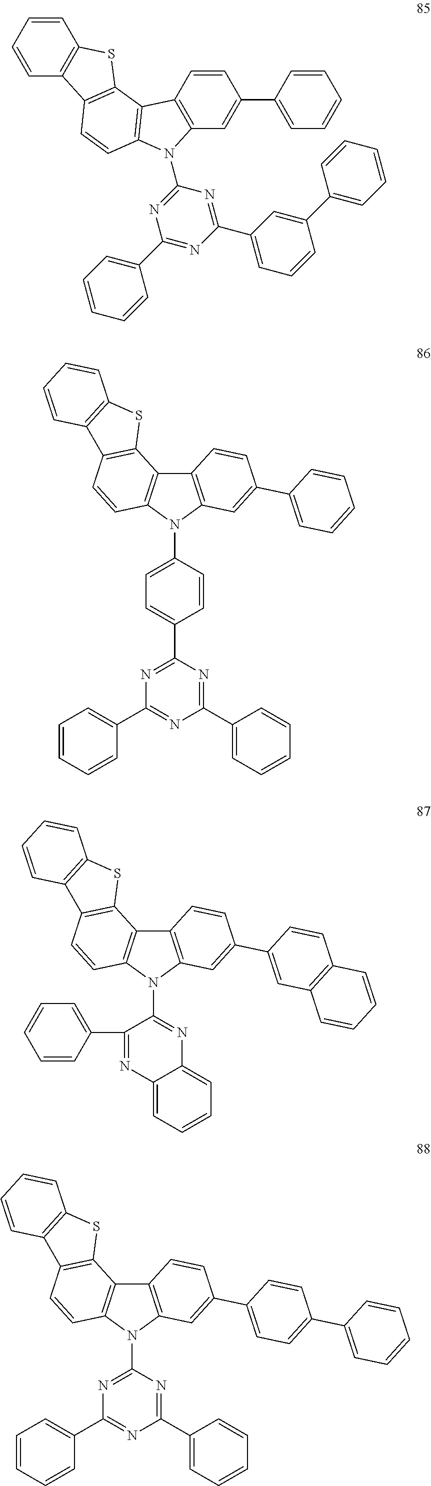 [SMILES: c1ccc(-c2cccc(-c3nc(-c4ccccc4)nc(-n4c5cc(-c6ccccc6)ccc5c5c6sc7ccccc7c6ccc54)n3)c2)cc1 ; c1ccc(-c2ccc3c4c5sc6ccccc6c5ccc4n(-c4ccc(-c5nc(-c6ccccc6)nc(-c6ccccc6)n5)cc4)c3c2)cc1 ; c1ccc(-c2nc3ccccc3nc2-n2c3cc(-c4ccc5ccccc5c4)ccc3c3c4sc5ccccc5c4ccc32)cc1 ; c1ccc(-c2ccc(-c3ccc4c5c6sc7ccccc7c6ccc5n(-c5nc(-c6ccccc6)nc(-c6ccccc6)n5)c4c3)cc2)cc1]

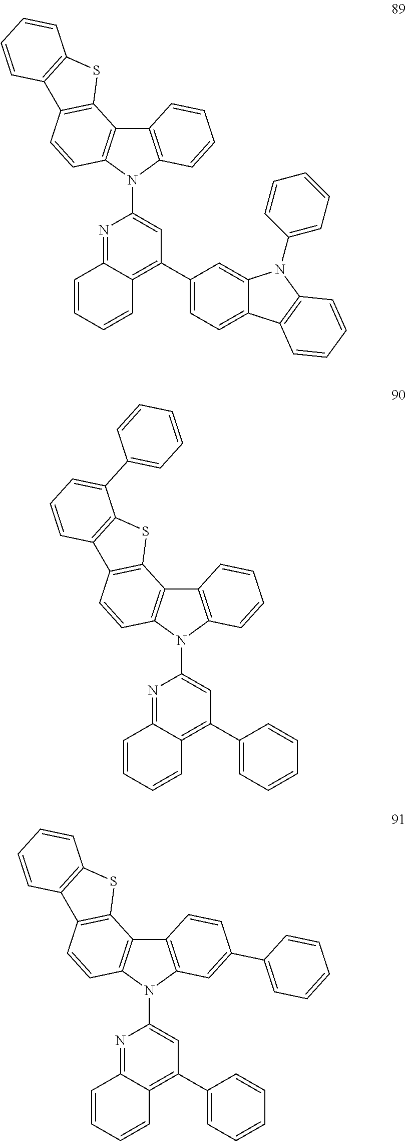 [SMILES: c1ccc(-n2c3ccccc3c3ccc(-c4cc(-n5c6ccccc6c6c7sc8ccccc8c7ccc65)nc5ccccc45)cc32)cc1 ; c1ccc(-c2cc(-n3c4ccccc4c4c5sc6c(-c7ccccc7)cccc6c5ccc43)nc3ccccc23)cc1 ; c1ccc(-c2ccc3c4c5sc6ccccc6c5ccc4n(-c4cc(-c5ccccc5)c5ccccc5n4)c3c2)cc1]

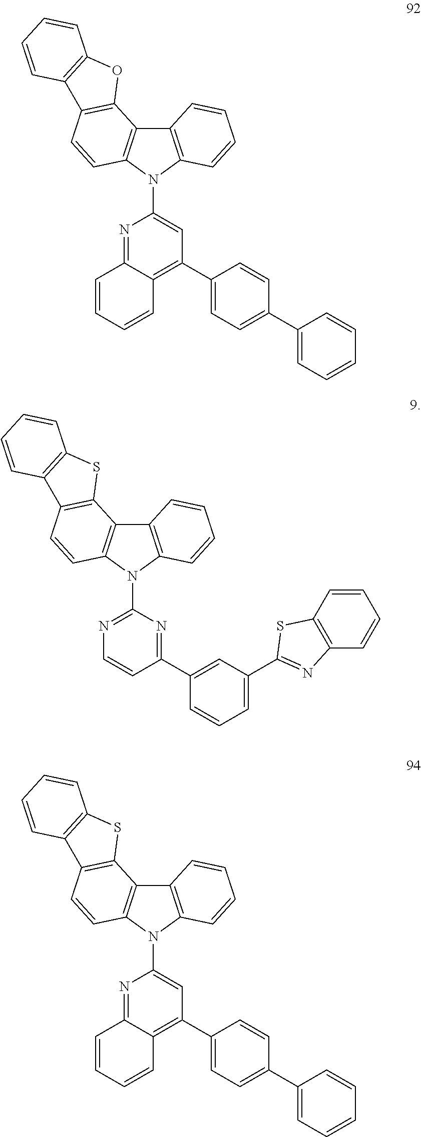 [SMILES: c1ccc(-c2ccc(-c3cc(-n4c5ccccc5c5c6oc7ccccc7c6ccc54)nc4ccccc34)cc2)cc1 ; c1cc(-c2ccnc(-n3c4ccccc4c4c5sc6ccccc6c5ccc43)n2)cc(-c2nc3ccccc3s2)c1 ; c1ccc(-c2ccc(-c3cc(-n4c5ccccc5c5c6sc7ccccc7c6ccc54)nc4ccccc34)cc2)cc1]

The compound of formula 1 included in an electron transport material according to the present invention can be prepared by known methods to one skilled in the art, and can be prepared, for example, according to the following reaction scheme 1:

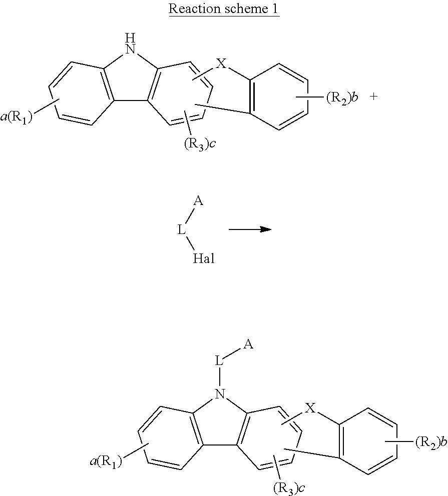

wherein

A, L, X, R₁ to R₃, a, b, and c are as defined in formula 1; and Hal represents a halogen.

The present invention further provides an electron transport material comprising the compound of formula 1, and an organic EL device comprising the electron transport material. An electron transport material can be comprised of the compound of formula 1 alone, or can be a mixture or composition for an electron transport layer which further comprises conventional materials generally included in electron transport materials.

The present invention further provides an organic EL device comprising an electron transport material of the present invention as another embodiment.

The organic EL device may further comprise a reducing dopant. The reducing dopant may be one or more selected from the group consisting of an alkaline metal, an alkaline earth metal, a rare-earth metal, an oxide of an alkaline metal, a halide of an alkaline metal, an oxide of an alkaline earth metal, a halide of an alkaline earth metal, an oxide of a rare-earth metal, a halide of a rare-earth metal, an organic complex of an alkaline metal, an organic complex of an alkaline earth metal, and an organic complex of a rare-earth metal. The specific examples of the reducing dopant may be lithium quinolate, sodium quinolate, cesium quinolate, potassium quinolate, LiF, NaCl, CsF, Li₂O, BaO, BaF₂, etc., but are not limited thereto. The reducing dopant may be included in an organic EL device as a combination with electron transport materials and can form a separate layer from electron transport materials.

The organic EL device of the present invention comprises an anode, a cathode, and at least one organic layer between the two electrodes. The organic layer comprises a light-emitting layer which contains host and dopant compounds. A light-emitting layer means a layer emitting light and may be a single layer or multi-layers having two or more layers. The doping concentration of dopant compounds to host compounds in a light-emitting layer is preferably less than 20 wt %.

The organic EL device of the present invention may further include at least one compound selected from the group consisting of arylamine-based compounds and styrylarylamine-based compounds in the organic layer.

In the organic EL device of the present invention, an organic layer may further comprise at least one metal selected from the group consisting of metals of Group 1, metals of Group 2, transition metals of the 4^{th} period, transition metals of the 5^{th} period, lanthanides, and organic metals of d-transition elements of the Periodic Table, or at least one complex compound comprising the metal.

Preferably, in the organic EL device of the present invention, at least one layer (hereinafter, “a surface layer”) selected from a chalcogenide layer, a metal halide layer, and a metal oxide layer may be placed on an inner surface(s) of one or both electrode(s). Specifically, it is preferred that a chalcogenide (including oxides) layer of silicon or aluminum is placed on an anode surface of a light-emitting medium layer, and a metal halide layer or metal oxide layer is placed on a cathode surface of an electroluminescent medium layer. The surface layer provides operating stability for the organic EL device. Preferably, the chalcogenide includes SiOₓ(1≦X≦2), AlOₓ(1≦X≦1.5), SiON, SiAION, etc.; the metal halide includes LiF, MgF₂, CaF₂, a rare earth metal fluoride, etc.; and the metal oxide includes Cs₂O, Li₂O, MgO, SrO, BaO, CaO, etc.

A hole injection layer (HIL), a hole transport layer (HTL), an electron blocking layer (EBL), or their combinations can be used between an anode and a light-emitting layer. A hole injection layer may be multi-layers in order to lower a hole injection barrier (or hole injection voltage) from an anode to a hole transport layer or an electron blocking layer, wherein each of the multi-layers simultaneously may use two compounds. A hole transport layer or an electron blocking layer may also be multi-layers.

An electron buffer layer, a hole blocking layer (HBL), an electron transport layer (ETL), an electron injection layer (EIL), or their combinations can be used between a light-emitting layer and a cathode. An electron buffer layer may be multi-layers in order to control the injection of an electron and improve interface properties between a light-emitting layer and an electron injection layer, wherein each of the multi-layers simultaneously may use two compounds. A hole blocking layer or an electron transport layer may also be multi-layers, wherein each of the multi-layers may use a multi-component of compounds. Preferably, in the organic EL device of the present invention, a mixed region of an electron transport compound and a reductive dopant, or a mixed region of a hole transport compound and an oxidative dopant may be placed on at least one surface of a pair of electrodes. In this case, an electron transport compound is reduced to an anion, and thus it becomes easier to inject and transport electrons from the mixed region to a light-emitting medium. Furthermore, a hole transport compound is oxidized to a cation, and thus it becomes easier to inject and transport holes from the mixed region to a light-emitting medium. Herein, an electron transport compound may be conventional electron transport compounds other than the compound of formula I which is used in an electron transport material of the present invention. Preferably, an oxidative dopant includes various Lewis acids and acceptor compounds; and a reductive dopant includes those recited above. A reductive dopant layer may be employed as a charge-generating layer to prepare an organic EL device having two or more light-emitting layers and emitting white light.

In order to form each layer constituting the organic EL device of the present invention, dry film-forming methods, such as vacuum deposition, sputtering, plasma, ion plating methods, etc., or wet film-forming methods, such as spin coating, dip coating, flow coating methods, etc., can be used.

When using a wet film-forming method, a thin film is formed by dissolving or dispersing the material constituting each layer in suitable solvents, such as ethanol, chloroform, tetrahydrofuran, dioxane, etc. The solvents are not specifically limited as long as the material constituting each layer is soluble or dispersible in the solvents, which do not cause any problems in forming layers.

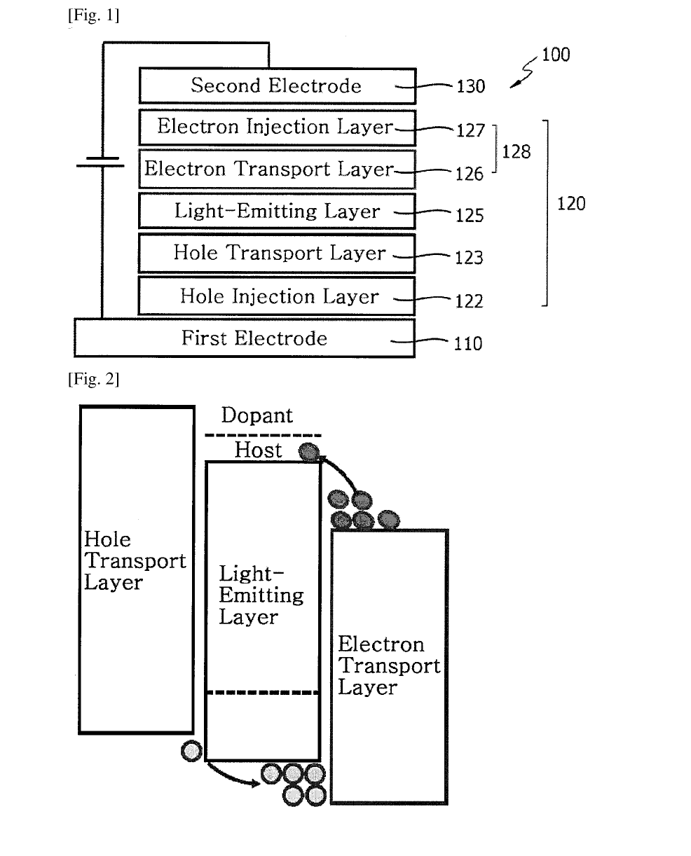

Hereinafter, referring to FIG. 1, the constitution of an organic EL device of the present invention will be described in detail.

FIG. 1 shows the typical cross-sectional diagram of an organic EL device comprising an electron transport layer comprising an electron transport material according to one embodiment of the present invention.

Referring to FIG. 1, the organic EL device (100) comprises a first electrode (110), an organic layer (120) formed on the first electrode (110), and a second electrode (130) which is opposite to the first electrode (110) and is formed on the organic layer (120).

The first electrode (110) may be an anode and the second electrode (130) may be a cathode.

The organic layer (120) comprises a hole injection layer (122), a hole transport layer (123) formed on the hole injection layer (122), a light-emitting layer (125) formed on the hole transport layer (123), and an electron transport zone (128) formed on the light-emitting layer (125), wherein the electron transport zone (128) comprises an electron transport layer (126) formed on the light-emitting layer (125) and an electron injection layer (127) formed on the electron transport layer (126). Each of the hole injection layer (122), the hole transport layer (123), the light-emitting layer (125), the electron transport layer (126), and the electron injection layer (127) may be a single layer or multi-layers having two or more layers.

The light-emitting layer (125) can be formed by using host and dopant compounds. The host and dopant compounds are not specifically limited and can be suitably selected from known compounds.

The electron transport zone (128) comprises an electron transport material of the present invention. Furthermore, FIG. 1 shows that the electron transport zone (128) comprises the electron transport layer (126) and the electron injection layer (127), but may comprise the electron transport layer (126) alone. The electron transport material of the present invention is preferably included in the electron transport layer of the electron transport zone.

The organic EL device of FIG. 1 is merely one embodiment to sufficiently explain the elements of the present invention to one skilled in the art, but the present invention is not limited thereto and can be specified to other embodiments. For example, in the organic EL device of FIG. 1, any one element such as a hole injection layer, except for a light-emitting layer and an electron transport zone, may be omitted. Furthermore, optional elements may be added to the device. The example of one element to be added may be an electron buffer layer.

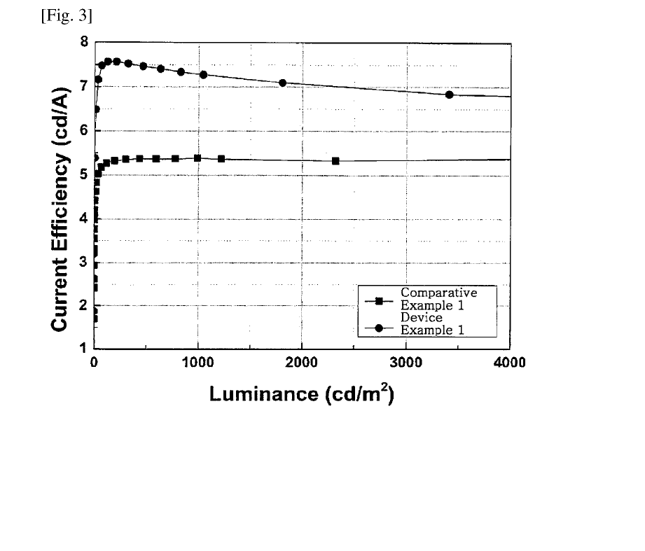

FIG. 2 briefly shows the relationship of energy gap between the layers disposed in an organic EL device according to one embodiment of the present invention.

In FIG. 2, a hole transport layer, a light-emitting layer, and an electron transport layer are sequentially stacked up, and electrons from a cathode are injected to the light-emitting layer via the electron transport layer. The LUMO energy value of the electron transport layer is higher than the LUMO energy values of host and dopant compounds in the light-emitting layer. Furthermore, as depicted in FIG. 2, even in the case where there is a big barrier between the light-emitting layer and the electron transport layer, when using an electron transport material according to the present invention, the organic EL device has fast electron current property, and thereby has lower driving voltage and higher efficiency.

Hereinafter, the compounds of the present invention, the preparation method thereof, and the luminous properties of devices comprising the compounds as an electron transport material will be explained in detail with reference to the representative compounds of the present invention.

### EXAMPLE 1

### Preparation of Compound 3

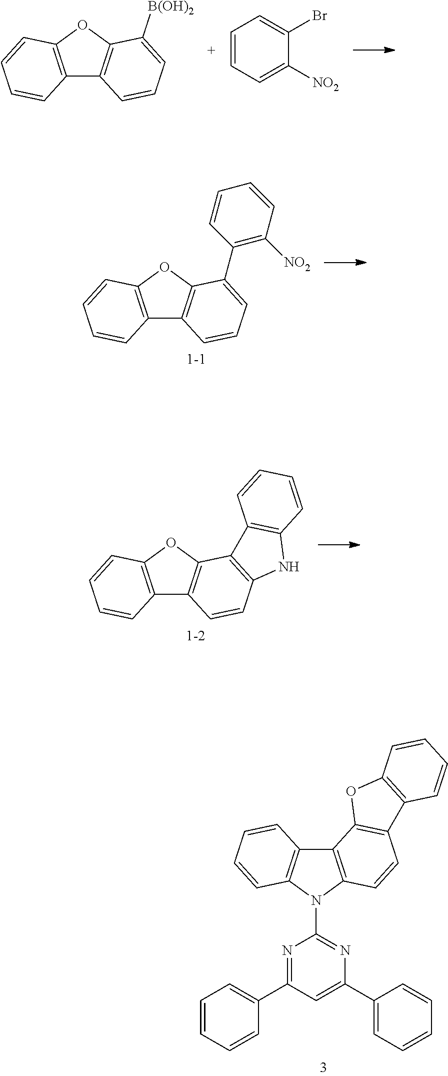 [SMILES: OB(O)c1cccc2c1oc1ccccc12 ; O=[N+]([O-])c1ccccc1Br ; O=[N+]([O-])c1ccccc1-c1cccc2c1oc1ccccc12 ; c1ccc2c(c1)[nH]c1ccc3c4ccccc4oc3c12 ; c1ccc(-c2cc(-c3ccccc3)nc(-n3c4ccccc4c4c5oc6ccccc6c5ccc43)n2)cc1]

### Preparation of Compound 1-1

After mixing 1-bromo-2-nitrobenzene (39.0 g, 0.19 mol), dibenzo[b,d]furan-4-yl boronic acid (45.0 g, 0.21 mol), tetrakis(triphenylphosphine)palladium(O) (Pd(PPh₃)₄) (11.1 g, 0.0096 mol), aqueous 2M K₂CO₃ solution (290.0 mL), ethanol (EtOH) (290.0 mL), and toluene (580.0 mL), the mixture was stirred for 4 hrs while heating to 120° C. Upon completing the reaction, the mixture was rinsed with distilled water and extracted with ethyl acetate (EA), the organic layer was dried with anhydrous MgSO₄, the solvent was removed by using a rotary evaporator, and the obtained product was purified through column chromatography to obtain compound 1-1 (47.0 g, 85%).

### Preparation of Compound 1-2

After mixing compound 1-1 (47.0 g, 0.16 mol), triethyl phosphite (600.0 mL), and 1,2-dichlorobenzene (300.0 mL), the mixture was stirred for 12 hrs while heating to 150° C. Upon completing the reaction, unreacted triethyl phosphite and 1,2-dichlorobenzene were removed by using a distillation equipment. The residue was rinsed with distilled water and extracted with EA, and the organic layer was dried with anhydrous MgSO₄. After removing the solvent by using a rotary evaporator, the obtained product was purified through column chromatography to obtain compound 1-2 (39.0 g, 81%).

### Preparation of Compound 3

NaH (1.9 mg, 42.1 mmol) was dissolved in dimethylformamide (DMF) and the mixture was stirred. After dissolving compound 1-2 (7.0 g, 27.2 mmol) in DMF, this solution was added to the stirred NaH solution and the resulting mixture was stirred for 1 hr. 2-Chloro-4,6-diphenylpyrimidine (8.7 g, 32.6 mmol) was dissolved in DMF and was stirred.

The mixture which was stirred for 1 hr in the previous step was added to the diphenylpyrimidine solution, and the resulting mixture was stirred at room temperature for 24 hrs. Upon completing the reaction, the resulting solid was filtered, rinsed with EA, and purified through column chromatography to obtain compound 3 (3.5 g, 25%).

### EXAMPLE 2

### Preparation of Compound 10

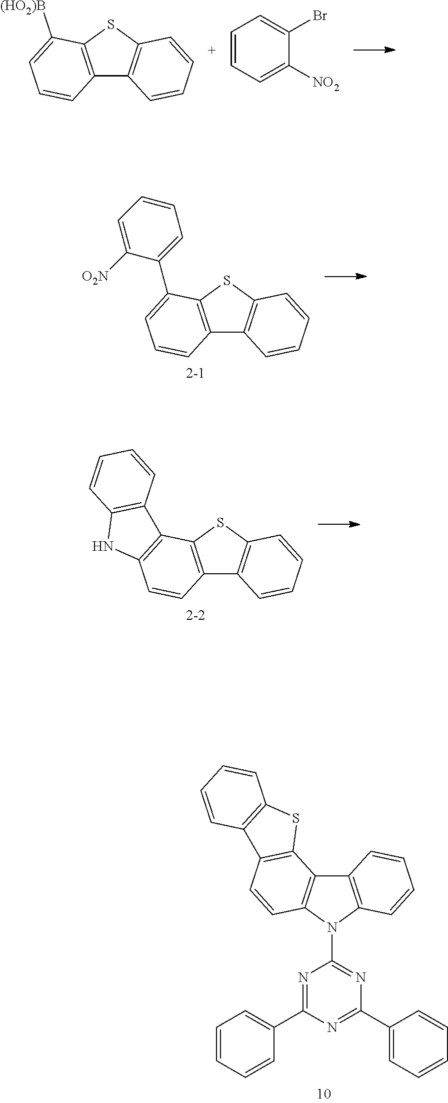

### Preparation of Compound 2-1

Compound 2-1 (10.0 g, 32.74 mmol, 74.68%) was produced in the same manner as in the preparation of compound 1-1 by using dibenzo[b,d]thiophene-4-yl boronic acid (10.0 g, 43.84 mmol).

### Preparation of Compound 2-2

Compound 2-2 (7.0 g, 25.60 mmol, 78.19%) was produced in the same manner as in the preparation of compound 1-2 by using compound 2-1 (10.0 g, 32.74 mmol).

### Preparation of Compound 10

Compound 10 (5.6 g, 40%) was produced in the same manner as in the preparation of compound 3 by using compound 2-2 (7.0 g, 25.6 mmol) and 2-chloro-4,6-diphenyl-1,3,5-triazine (8.7 g, 32.6 mmol).

### EXAMPLE 3

### Preparation of compound 22

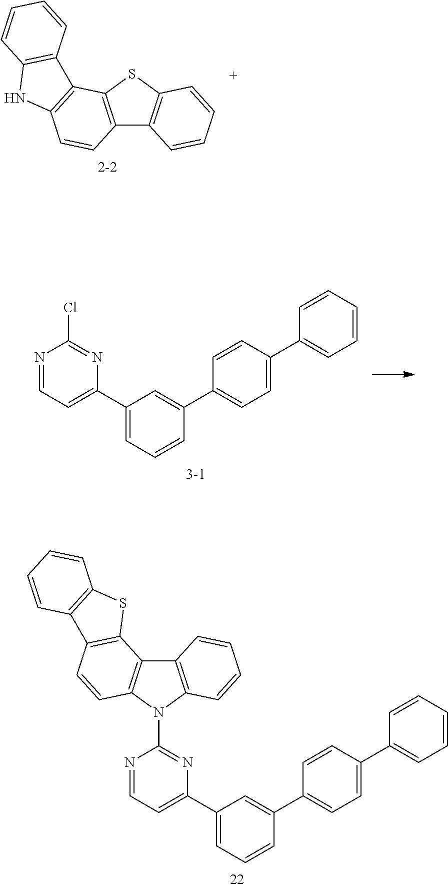 [SMILES: c1ccc2c(c1)[nH]c1ccc3c4ccccc4sc3c12 ; Clc1nccc(-c2cccc(-c3ccc(-c4ccccc4)cc3)c2)n1 ; c1ccc(-c2ccc(-c3cccc(-c4ccnc(-n5c6ccccc6c6c7sc8ccccc8c7ccc65)n4)c3)cc2)cc1]

Target compound 22 (5.3 g, 49%) was produced in the same manner as in the preparation of compound 3 by using compound 2-2 (7.0 g, 25.6 mmol) and compound 3-1 (8.2 g, 32.6 mmol).

Compounds 1 to 72 were produced in the same manner as in Examples 1 to 3. Specific data of the physical properties of the representative compounds among the produced compounds are provided in Table 1 below.

TABLE 1

| | | M.P. | | | MS/EIMS |
|---|---|---|---|---|---|
| Compound | Yield (%) | (° C.) | UV (nm) | PL (nm) | (Found) |
| 3 | 25 | 260 | 358 | 471 | 488.5 |
| 4 | 30 | 259 | 336 | 463 | 686.9 |
| 6 | 26 | 350 | 356 | 429 | 581.7 |
| 7 | 46 | 225 | 338 | 482 | 504.3 |
| 8 | 78 | 312 | 344 | 385 | 489.5 |
| 9 | 67 | 249 | 324 | 458 | 610.7 |
| 10 | 40 | 324 | 352 | 482 | 505.7 |
| 11 | 45 | 255 | 334 | 451 | 581.7 |
| 12 | 89 | 275 | 320 | 456 | 580.7 |
| 13 | 72 | 267 | 334 | 459 | 610.7 |
| 15 | 46 | 270 | 344 | 471 | 593.7 |
| 18 | 42 | 288 | 370 | 475 | 745.9 |
| 19 | 28 | 323 | N/A | N/A | 746.8 |
| 20 | 39 | 320 | 325 | 516 | 581.7 |
| 21 | 38 | 198 | 317 | 461 | 504.6 |
| 22 | 49 | 274 | 322 | 491 | 580.7 |
| 24 | 49 | 284 | 368 | 474 | 669.8 |
| 25 | 23 | 270 | 324 | 456 | 763 |
| 26 | 26 | 245 | 300 | 460 | 656.8 |
| 27 | 52 | 241 | 294 | 464 | 581.7 |
| 28 | 42 | 328 | 343 | 481 | 656.8 |
| 29 | 32 | 294 | 296 | 467 | 655.2 |
| 31 | 34 | 294 | N/A | N/A | 656.8 |
| 32 | 60 | 280 | 294 | 468 | 593.7 |
| 34 | 46 | 324 | 324 | 495 | 589.7 |
| 35 | 82 | 250 | 356 | 448 | 669.8 |
| 38 | 30 | 293 | 344 | 469 | 669.8 |
| 39 | 23 | 238 | 362 | 429 | 593.7 |
| 40 | 44 | 357 | 322 | 460 | 655.8 |
| 44 | 48 | 278 | 344 | 395 | 580.7 |
| 47 | 48 | 221 | 334 | 396 | 656.8 |
| 49 | 16 | 347 | 324 | 525 | 669.9 |
| 50 | 34 | 410 | 258 | 324 | 670.8 |
| 51 | 36 | 300 | 258 | 487 | 686.9 |
| 52 | 57 | 261 | 344 | 431 | 593.7 |
| 55 | 23 | 300 | 336 | 458 | 580.7 |
| 64 | 24 | 275 | 344 | 467 | 610.8 |
| 67 | 50 | 305 | 350 | 502 | 656.8 |
| 68 | 66 | 305 | 306 | 407 | 637.8 |
| 69 | 22 | 238 | 304 | 465 | 636.8 |
| 70 | 27 | 274 | 308 | 463 | 620.7 |

COMPARATIVE EXAMPLE 1

### Production of a Blue Light-Emitting Organic EL Device which is not in Accordance with the Present Invention

An OLED device comprising an organic compound for an electron transport material which is not in accordance with the present invention was produced as follows: A transparent electrode indium tin oxide (ITO) thin film (10 Ω/sq) on a glass substrate for an OLED device (GEOMATEC CO., LTD., Japan) was subjected to an ultrasonic washing by sequentially using acetone, ethanol, and distilled water, and was then stored in isopropanol. Next, the ITO substrate was mounted on a substrate holder of a vacuum vapor depositing apparatus. N⁴,N⁴′-diphenyl-N⁴,N⁴′-bis(9-phenyl-9H-carbazol-3-yl)-[1,1′-biphenyl]-4,4′-diamine (compound HI-1) was introduced into a cell of the vacuum vapor depositing apparatus, and the pressure in the chamber of the apparatus was then controlled to 10⁻⁶ torr. Thereafter, an electric current was applied to the cell to evaporate the introduced material, thereby forming a hole injection layer 1 having a thickness of 60 nm on the ITO substrate. 1,4,5,8,9,12-hexaazatriphenylene-hexacarbonitrile (compound HI-2) was then introduced into another cell of the vacuum vapor depositing apparatus, and an electric current was applied to the cell to evaporate the introduced material, thereby forming a hole injection layer 2 having a thickness of 5 nm on hole injection layer 1. N-([1,1′-biphenyl]-4-yl)-9,9-dimethyl-N-(4-(9-phenyl-9H-carbazol-3-yl)phenyl)-9H-fluorene-2-amine (compound HT-1) was introduced into another cell of the vacuum vapor depositing apparatus. Thereafter, an electric current was applied to the cell to evaporate the introduced material, thereby forming a hole transport layer 1 having a thickness of 20 nm on hole injection layer 2. N,N-di([1,1′-biphenyl]-4-yl)-4′-(9H-carbazol-9-yl)-[1,1′-biphenyl]-4-amine (compound HT-2) was then introduced into another cell of the vacuum vapor depositing apparatus, and an electric current was applied to the cell to evaporate the introduced material, thereby forming a hole transport layer 2 having a thickness of 5 nm on hole transport layer 1. After forming the hole injection layer and the hole transport layer, a light-emitting layer was then deposited as follows. Compound BH-1 as a host compound was introduced into one cell of the vacuum vapor depositing apparatus and compound BD-1 as a dopant was introduced into another cell of the apparatus. The two materials were evaporated at a different rate and the dopant was deposited in a doping amount of 2 wt %, based on the total weight of the host and dopant, to form a light-emitting layer having a thickness of 20 nm on the hole transport layer. Next, compound ETL-1 as an electron transport material was evaporated on one cell to form an electron transport layer having a thickness of 33 nm on the light-emitting layer. After depositing lithium quinolate having a thickness of 4 nm as an electron injection layer on the electron transport layer, an Al cathode having a thickness of 80 nm was then deposited by another vacuum vapor deposition apparatus on the electron injection layer. Thus, an OLED device was produced. All the materials used for producing the OLED device were purified by vacuum sublimation at 10⁻⁶ torr prior to use.

The current efficiency vs. luminance values of the OLED device produced above are shown in a graph in FIG. 3. Furthermore, the driving voltage at a luminance of 1,000 nit, the luminous efficiency, and the CIE color coordinate of the OLED device produced in Comparative Example 1 are provided in Table 2 below.

### DEVICE EXAMPLES 1 to 12

### Production of a Blue Light-Emitting Organic EL Device According to the Present Invention

OLED devices were produced in the same manner as in Comparative Example 1, except that an electron transport material was changed to the compounds as shown in Table 2 below. Evaluation results of the OLED devices produced in each of Device Examples 1 to 12 are provided in Table 2 below. Furthermore, the current efficiency vs. luminance values of the OLED device produced in Device Example 1 are shown in a graph in FIG. 3.

### COMPARATIVE EXAMPLE 2

### Production of a Blue Light-Emitting Organic EL Device which is not in Accordance with the Present Invention

An OLED device was produced in the same manner as in Comparative Example 1, except that compound ETL-2 was used as an electron transport material. Evaluation results of the OLED device produced in Comparative Example 2 are provided in Table 2 below.

TABLE 2

| | Electron | Driving | Luminous | Color | Color | | |
|---|---|---|---|---|---|---|---|
| | Transport | Voltage | Efficiency | Coordinate | Coordinate | LUMO | HOMO |
| | Material | (V) | (cd/A) | (x) | (y) | (eV) | (eV) |
| CE 1 | ETL-1 | 5.0 | 5.4 | 0.141 | 0.142 | 1.81 | 5.12 |
| CE 2 | ETL-2 | 5.4 | 5.5 | 0.138 | 0.101 | 1.96 | 5.37 |
| DE 1 | ETL-3 | 4.2 | 7.3 | 0.138 | 0.103 | 1.92 | 5.40 |
| | (Compound 77) | | | | | | |
| DE 2 | ETL-4 | 4.2 | 7.5 | 0.138 | 0.107 | 1.92 | 5.35 |
| | (Compound 78) | | | | | | |
| DE 3 | ETL-5 | 4.3 | 7.3 | 0.138 | 0.105 | 1.93 | 5.35 |
| | (Compound 79) | | | | | | |
| DE 4 | ETL-6 | 5.0 | 6.6 | 0.138 | 0.103 | 1.90 | 5.60 |
| | (Compound 80) | | | | | | |
| DE 5 | ETL-7 | 4.4 | 6.7 | 0.138 | 0.109 | 1.98 | 5.30 |
| | (Compound 76) | | | | | | |
| DE 6 | ETL-8 | 4.5 | 6.3 | 0.138 | 0.108 | 1.99 | 5.30 |
| | (Compound 75) | | | | | | |
| DE 7 | ETL-9 | 4.6 | 5.7 | 0.138 | 0.106 | 1.95 | 5.50 |
| | (Compound 73) | | | | | | |
| DE 8 | ETL-10 | 4.4 | 6.9 | 0.138 | 0.105 | 1.97 | 5.59 |
| | (Compound 10) | | | | | | |
| DE 9 | ETL-11 | 4.7 | 6.3 | 0.138 | 0.111 | 1.82 | 5.34 |
| | (Compound 33) | | | | | | |
| DE 10 | ETL-12 | 4.4 | 6.4 | 0.138 | 0.115 | 2.06 | 5.54 |
| | (Compound 56) | | | | | | |
| DE 11 | ETL-13 | 4.9 | 7.4 | 0.138 | 0.112 | 1.86 | 5.37 |
| | (Compound 66) | | | | | | |
| DE 12 | ETL-14 | 5.0 | 6.2 | 0.138 | 0.106 | 1.97 | 5.45 |
| | (Compound 93) | | | | | | |

(In Table 2 above, “CE” and “DE” mean Comparative Example and Device Example, respectively.)

Based on Table 2 above, an electron transport layer (ETL) of the present invention has fast electron current property, and thus Device Examples 1 to 12 provide high efficiency compared with Comparative Examples 1 and 2. Furthermore, from FIG. 3, it can be seen that the OLED device of Device Example 1 has high current efficiency over the entire luminance area compared with the OLED device of Comparative Example 1. Upon comparing Comparative Example 2 with Device Example 2, the compound used in an electron transport material of Comparative Example 2 has the structure in which a carbazole ring is bonded to a benzofuran ring via a direct bond, and thus its dihedral angle is relatively larger than that of the compound used in an electron transport material of Device Example 2 wherein a benzofuran ring is directly fused with a carbazole ring. Thus, it is thought that electron injection does not go on smoothly in Comparative Example 2 compared with Device Example 2, and thus Comparative Example 2 shows high driving voltage and low luminous efficiency.

TABLE 3
Compounds used in the Comparative Examples and Device Examples

| | |
|---|---|
| Hole Injection Layer/ Hole Transport Layer | 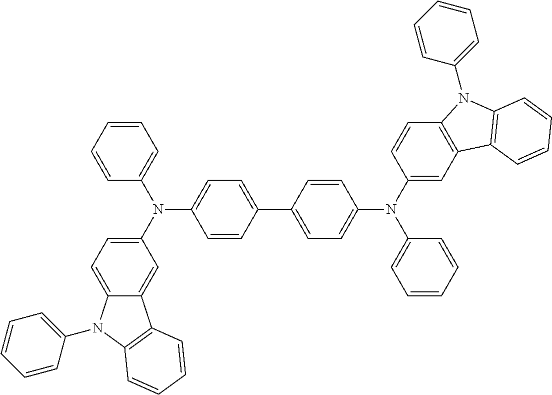 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1] HI-1 |
| | 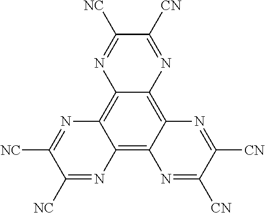 [SMILES: N#Cc1nc2c3nc(C#N)c(C#N)nc3c3nc(C#N)c(C#N)nc3c2nc1C#N] HI-2 |
| | 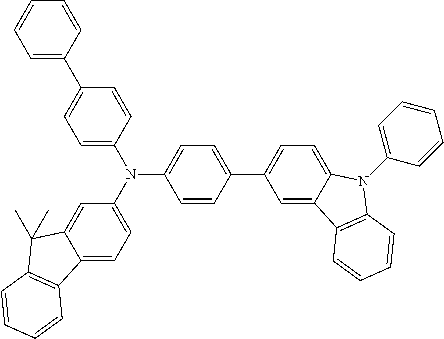 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21] HT-1 |
| | 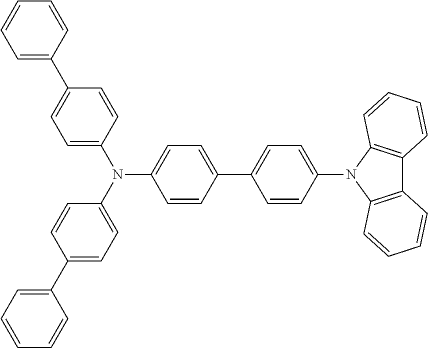 [SMILES: c1ccc(-c2ccc(N(c3ccc(-c4ccccc4)cc3)c3ccc(-c4ccc(-n5c6ccccc6c6ccccc65)cc4)cc3)cc2)cc1] HT-2 |
| Light- Emitting Layer | 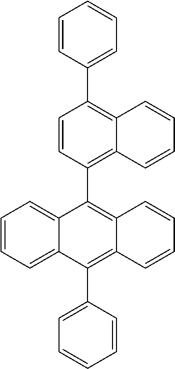 [SMILES: c1ccc(-c2ccc(-c3c4ccccc4c(-c4ccccc4)c4ccccc34)c3ccccc23)cc1] BH-1 |
| | 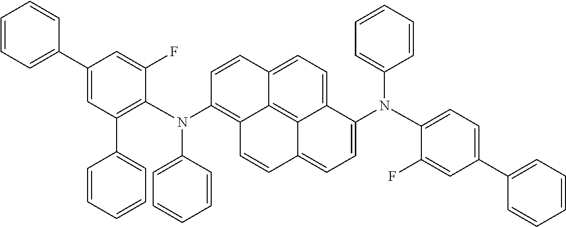 [SMILES: Fc1cc(-c2ccccc2)ccc1N(c1ccccc1)c1ccc2ccc3c(N(c4ccccc4)c4c(F)cc(-c5ccccc5)cc4-c4ccccc4)ccc4ccc1c2c43] BD-1 |
| Electron Transport Layer/ Electron Injection Layer | 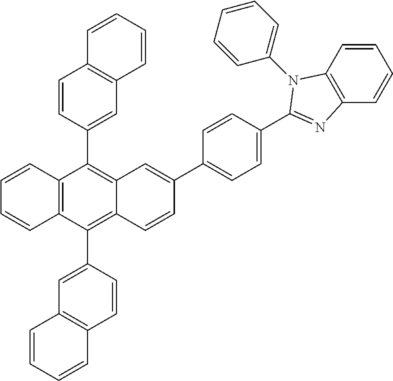 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1] ETL-1 |
| | 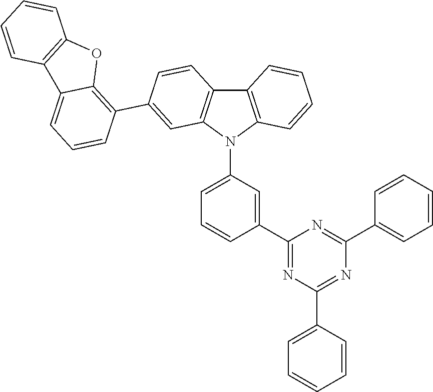 [SMILES: c1ccc(-c2nc(-c3ccccc3)nc(-c3cccc(-n4c5ccccc5c5ccc(-c6cccc7c6oc6ccccc67)cc54)c3)n2)cc1] ETL-2 |
| | 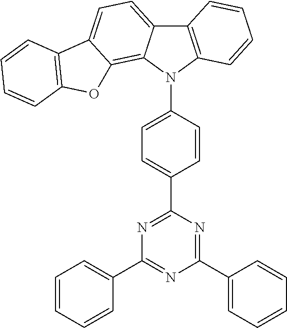 [SMILES: c1ccc(-c2nc(-c3ccccc3)nc(-c3ccc(-n4c5ccccc5c5ccc6c7ccccc7oc6c54)cc3)n2)cc1] ETL-3 |
| | 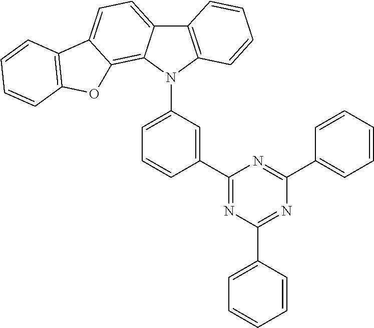 [SMILES: c1ccc(-c2nc(-c3ccccc3)nc(-c3cccc(-n4c5ccccc5c5ccc6c7ccccc7oc6c54)c3)n2)cc1] ETL-4 |
| | 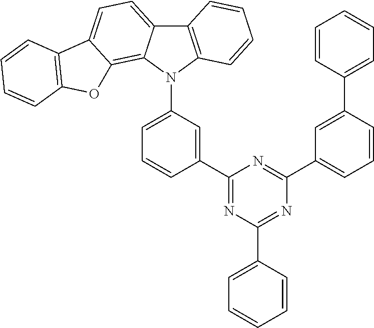 [SMILES: c1ccc(-c2cccc(-c3nc(-c4ccccc4)nc(-c4cccc(-n5c6ccccc6c6ccc7c8ccccc8oc7c65)c4)n3)c2)cc1] ETL-5 |
| | 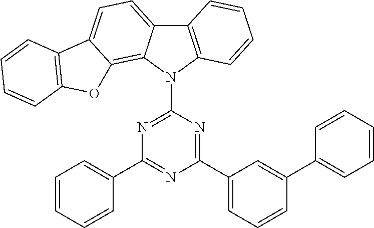 [SMILES: c1ccc(-c2cccc(-c3nc(-c4ccccc4)nc(-n4c5ccccc5c5ccc6c7ccccc7oc6c54)n3)c2)cc1] ETL-6 |
| | 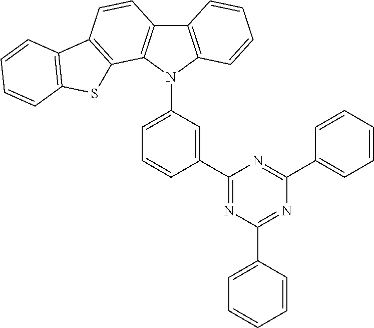 [SMILES: c1ccc(-c2nc(-c3ccccc3)nc(-c3cccc(-n4c5ccccc5c5ccc6c7ccccc7sc6c54)c3)n2)cc1] ETL-7 |
| | 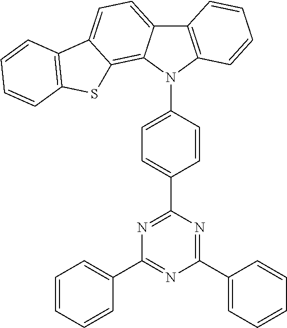 [SMILES: c1ccc(-c2nc(-c3ccccc3)nc(-c3ccc(-n4c5ccccc5c5ccc6c7ccccc7sc6c54)cc3)n2)cc1] ETL-8 |
| | 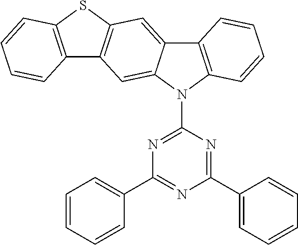 [SMILES: c1ccc(-c2nc(-c3ccccc3)nc(-n3c4ccccc4c4cc5sc6ccccc6c5cc43)n2)cc1] ETL-9 |
| | 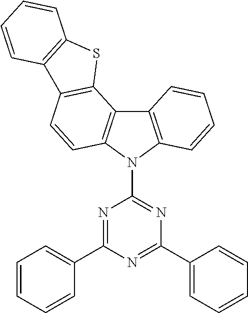 [SMILES: c1ccc(-c2nc(-c3ccccc3)nc(-n3c4ccccc4c4c5sc6ccccc6c5ccc43)n2)cc1] ETL-10 |
| | 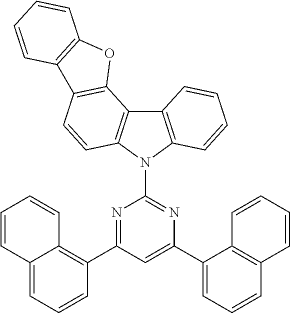 [SMILES: c1ccc2c(-c3cc(-c4cccc5ccccc45)nc(-n4c5ccccc5c5c6oc7ccccc7c6ccc54)n3)cccc2c1] ETL-11 |
| | 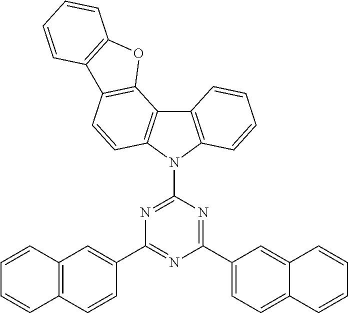 [SMILES: c1ccc2cc(-c3nc(-c4ccc5ccccc5c4)nc(-n4c5ccccc5c5c6oc7ccccc7c6ccc54)n3)ccc2c1] ETL-12 |
| | 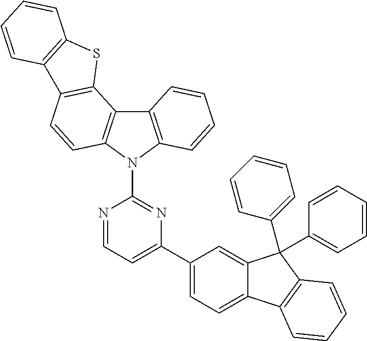 [SMILES: c1ccc(C2(c3ccccc3)c3ccccc3-c3ccc(-c4ccnc(-n5c6ccccc6c6c7sc8ccccc8c7ccc65)n4)cc32)cc1] ETL-13 |
| | 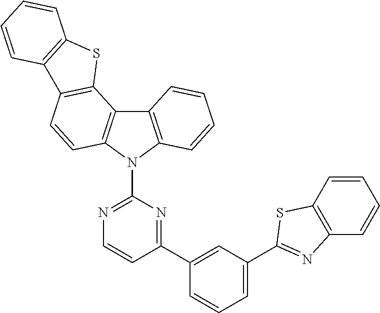 [SMILES: c1cc(-c2ccnc(-n3c4ccccc4c4c5sc6ccccc6c5ccc43)n2)cc(-c2nc3ccccc3s2)c1] ETL-14 |
| | 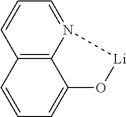 EIL-1 |

Characteristic feature of an electron transport material comprising the compound of the present invention.

The compound represented by formula 1 has the structure in which benzofuran or benzothiophene is fused to a carbazole derivative to form benzofurocarbazole or benzothienocarbazole.

The above structure is rigid by fusing a carbazole ring to a benzothiophene ring or a benzofuran ring, and thus has almost 0° of dihedral angle. According to this structure, relevant bulky groups have great intermolecular π-orbital overlap, and thus intermolecular charge transition becomes easier, and it is considered that if the intermolecular π-π stacking is reinforced, fast electron current property can be achieved through a coplanar structure. On the contrary, since the compounds used in the Comparative Examples have the structure in which a carbazole ring is bonded to a benzothiophene ring or a benzofuran ring via a direct bond, its dihedral angle has a deviation of about 36° which provides relatively random molecular orientation, and thereby resulting in several problems that electron current property deteriorates and efficiency is reduced. Therefore, an electron transport material comprising the compound according to the present invention can greatly contribute to the low driving voltage and high efficiency of an OLED device.

The data in Table 2 above were determined under the condition that the electron affinity of an electron transport layer (Ab) is higher than the electron affinity of a host (Ah, LUMO=1.6 eV), and the electron transport layers of the Device Examples according to the present invention have higher electron affinity than that of Comparative Example 1. LUMO (lowest unoccupied molecular orbital) energy value and HOMO (highest occupied molecular orbital) energy value have inherently negative numbers, but the LUMO energy value (A) and the HOMO energy value in the present invention are conveniently expressed as their absolute values. Furthermore, the comparison between LUMO energy values is based on their absolute values. The LUMO energy value and the HOMO energy value in the present invention are calculated by Density Functional Theory (DFT).

The respective electron transport layer and electron injection layer may be comprised of two or more layers. The LUMO energy value of the electron transport layer may be smaller than the LUMO energy value of a light-emitting layer. For example, the LUMO energy values of the electron transport layer and the light-emitting layer may be 1.9 eV and 1.6 eV, respectively. Thus, a difference between the LUMO energy values of the two layers may be 0.3 eV. If an electron transport layer has the LUMO energy value as described above, it is difficult to inject electrons to a light-emitting layer through the electron transport layer. However, an electron transport layer produced by using an electron transport material comprising the compound of formula 1 easily transports electrons to a light-emitting layer. Thus, the OLED device of the present invention has low driving voltage and high luminous efficiency.

LUMO energy values can be easily determined by using various known methods. Conventionally, LUMO energy values can be determined by using cyclic voltammetry or ultraviolet photoelectron spectroscopy (UPS). Thus, one skilled in the art can easily recognize an electron buffer layer, a host material, and an electron transport zone which satisfy the relationship of the LUMO energy values of the present invention and specifically embody the present invention. HOMO energy values can also be easily determined in the same manner as used for the LUMO energy values.

According to the present invention, as depicted in FIG. 2, although the devices according to the present invention have a big barrier between a light-emitting layer and an electron transport layer in the process of transporting electrons compared with the device of Comparative Example 1 (see LUMO energy value), the devices of the present invention have fast electron current property, and thus have lower driving voltage and higher efficiency than the device of Comparative Example 1. Furthermore, the compounds of the present invention have higher HOMO energy values than the comparative compounds, and thus efficiently restrict movement of excitons produced in a light-emitting layer and hole carriers as shown in FIG. 3. According to this, the compounds of the present invention are regarded as showing color coordinates being the nearest to pure blue compared with the comparative compounds.

[Reference numbers in the Figures]

| | |
|---|---|
| 100: Organic light-emitting device | 110: First electrode |
| 120: Organic layer | 122: Hole injection layer |
| 123: Hole transport layer | 125: Light-emitting layer |
| 126: Electron transport layer | 127: Electron injection layer |
| 128: Electron transport zone | 130: Second electrode |

1. An electron transport material comprising a compound represented by the following formula 1: 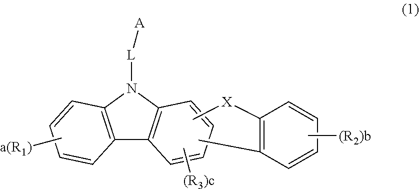
wherein
A represents a substituted or unsubstituted 5- to 30-membered heteroaryl group;
L represents a single bond, a substituted or unsubstituted (C6-C30)arylene group, or a substituted or unsubstituted 5- to 30-membered heteroarylene group;
X represents O or S;
R₁ and R₂ each independently represent a hydrogen, deuterium, a halogen, a cyano group, a substituted or unsubstituted (C1-C30)alkyl group, a substituted or unsubstituted (C6-C30)aryl group, a substituted or unsubstituted 5- to 30-membered heteroaryl group, a substituted or unsubstituted (C6-C30)aryl(C1-C30)alkyl group, a substituted or unsubstituted (C3-C30)cycloalkyl group, a substituted or unsubstituted (C1-C30)alkoxy group, a substituted or unsubstituted tri(C1-C30)alkylsilyl group, a substituted or unsubstituted tri(C6-C30)arylsilyl group, a substituted or unsubstituted di(C1-C30)alkyl(C6-C30)arylsilyl group, a substituted or unsubstituted (C1-C30)alkyldi(C6-C30)arylsilyl group, a substituted or unsubstituted mono- or di-(C1-C30)alkylamino group, a substituted or unsubstituted mono- or di-(C6-C30)arylamino group, or a substituted or unsubstituted (C1-C30)alkyl(C6-C30)arylamino group; or may be linked to an adjacent substituent(s) to form a substituted or unsubstituted mono- or polycyclic, (C3-C30) alicyclic or aromatic ring whose carbon atom(s) may be replaced with at least one hetero atom selected from nitrogen, oxygen, and sulfur;
R₃ represents a hydrogen, deuterium, a halogen, a cyano group, a substituted or unsubstituted (C1-C30)alkyl group, a substituted or unsubstituted (C6-C30)aryl group, or a substituted or unsubstituted 5- to 30-membered heteroaryl group; or may be linked to an adjacent substituent(s) to form a substituted or unsubstituted mono- or polycyclic, (C3-C30) alicyclic or aromatic ring whose carbon atom(s) may be replaced with at least one hetero atom selected from nitrogen, oxygen, and sulfur;
a and b each independently represent an integer of 1 to 4; where a or b is an integer of 2 or more, each of R₁ or each of R₂ may be the same or different;
c represents an integer of 1 to 2; where c is an integer of 2, each of R₃ may be the same or different; and
the heteroaryl(ene) group contains at least one hetero atom selected from B, N, O, S, Si, and P.

2. The electron transport material according to claim 1, wherein the compound of formula 1 is represented by one of the following formulae 2 to 7: 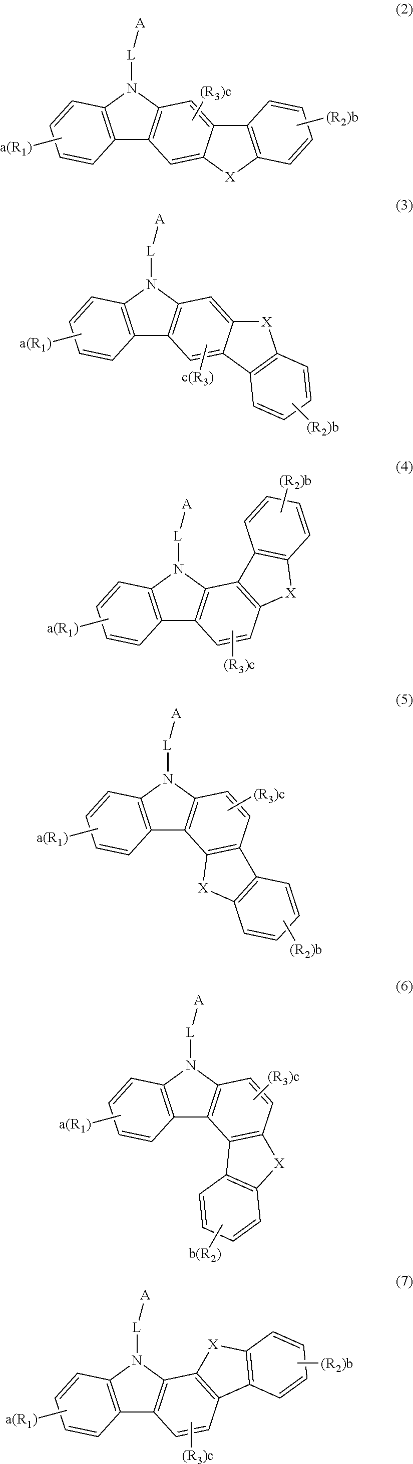
wherein A, L, R₁ to R₃, a, b, and c are as defined in claim 1.

3. The electron transport material according to claim 1, wherein the substituents of the substituted alkyl group, the substituted alkoxy, the substituted cycloalkyl group, the substituted aryl(ene) group, the substituted heteroaryl(ene) group, the substituted trialkylsilyl group, the substituted triarylsilyl group, the substituted dialkylarylsilyl group, the substituted alkyldiarylsilyl group, the substituted mono- or di-arylamino group, the substituted mono- or di-alkylamino group, the substituted alkylarylamino group, the substituted aralkyl group, and the substituted mono- or polycyclic, (C3-C30) alicyclic or aromatic ring in A, L, and R₁ to R₃ are each independently at least one selected from the group consisting of deuterium; a halogen; a cyano group; a carboxyl group; a nitro group; a hydroxyl group; a (C1-C30)alkyl group; a halo(C1-C30)alkyl group; a (C2-C30)alkenyl group; a (C2-C30)alkynyl group; a (C1-C30)alkoxy group; a (C1-C30)alkylthio group; a (C3-C30)cycloalkyl group; a (C3-C30)cycloalkenyl group; a 3- to 7-membered heterocycloalkyl group; a (C6-C30)aryloxy group; a (C6-C30)arylthio group; a 3- to 30-membered heteroaryl group which is unsubstituted or substituted with a (C1-C30)alkyl group or a (C6-C30)aryl group; a (C6-C30)aryl group which is unsubstituted or substituted with a (C1-C30)alkyl group or a 3- to 30-membered heteroaryl group; a (C6-C30)aryl group which is substituted with a tri(C1-C30)alkylsilyl group; a (C6-C30)aryl group which is substituted with a tri(C6-C30)arylsilyl group; a tri(C1-C30)alkylsilyl group; a tri(C6-C30)arylsilyl group; a di(C1-C30)alkyl(C6-C30)arylsilyl group; a (C1-C30)alkyldi(C6-C30)arylsilyl group; an amino group; a mono- or di-(C1-C30)alkylamino group; a mono- or di-(C6-C30)arylamino group; a (C1-C30)alkyl(C6-C30)arylamino group; a (C1-C30)alkylcarbonyl group; a (C1-C30)alkoxycarbonyl group; a (C6-C30)arylcarbonyl group; a di(C6-C30)arylboronyl group; a di(C1-C30)alkylboronyl group; a (C1-C30)alkyl(C6-C30)arylboronyl group; a (C6-C30)aryl(C1-C30)alkyl group; and a (C1-C30)alkyl(C6-C30)aryl group.

4. The electron transport material according to claim 1, wherein A represents a substituted or unsubstituted 5- to 20-membered heteroaryl group; L represents a single bond, a substituted or unsubstituted (C6-C20)arylene group, or a substituted or unsubstituted 5- to 20-membered heteroarylene group; X represents O or S; R₁ and R₂ each independently represent a hydrogen, a substituted or unsubstituted (C6-C20)aryl group, or a substituted or unsubstituted 5- to 20-membered heteroaryl group; R₃ represents a hydrogen; a and b each independently represent an integer of 1 to 2; and c represents 1.

5. The electron transport material according to claim 1, wherein A represents an unsubstituted 5- to 20-membered heteroaryl group, a 5- to 20-membered heteroaryl group substituted with a 5- to 20-membered heteroaryl group which is unsubstituted or substituted with a (C1-C20)alkyl group or a (C6-C20)aryl group, a 5- to 20-membered heteroaryl group substituted with a (C6-C20)aryl group, a 5- to 20-membered heteroaryl group substituted with a (C6-C20)aryl group substituted with a 5- to 20-membered heteroaryl group, a 5- to 20-membered heteroaryl group substituted with a (C6-C20)aryl group substituted with a tri(C1-C6)alkylsilyl group, a 5- to 20-membered heteroaryl group substituted with a (C6-C20)aryl group substituted with a tri(C6-C20)arylsilyl group, or a 5- to 20-membered heteroaryl group substituted with a (C1-C6)alkyl(C6-C20)aryl group; L represents a single bond, an unsubstituted (C6-C20)arylene group, or an unsubstituted 5- to 20-membered heteroarylene group; X represents O or S; R₁ and R₂ each independently represent a hydrogen, a (C6-C20)aryl group which is unsubstituted or substituted with a (C6-C12)aryl group, or a 5- to 20-membered heteroaryl group which is unsubstituted or substituted with a (C6-C20)aryl group; R₃ represents a hydrogen; a and b each independently represent an integer of 1 to 2; and c represents an integer of 1.

6. The electron transport material according to claim 1, wherein A represents a substituted or unsubstituted pyridine, a substituted or unsubstituted pyrimidine, a substituted or unsubstituted triazine, a substituted or unsubstituted pyrazine, a substituted or unsubstituted quinoline, a substituted or unsubstituted quinazoline, a substituted or unsubstituted quinoxaline, a substituted or unsubstituted naphthyridine, or a substituted or unsubstituted phenanthroline.

7. The electron transport material according to claim 1, wherein the compound represented by formula 1 is selected from the group consisting of the following compounds:

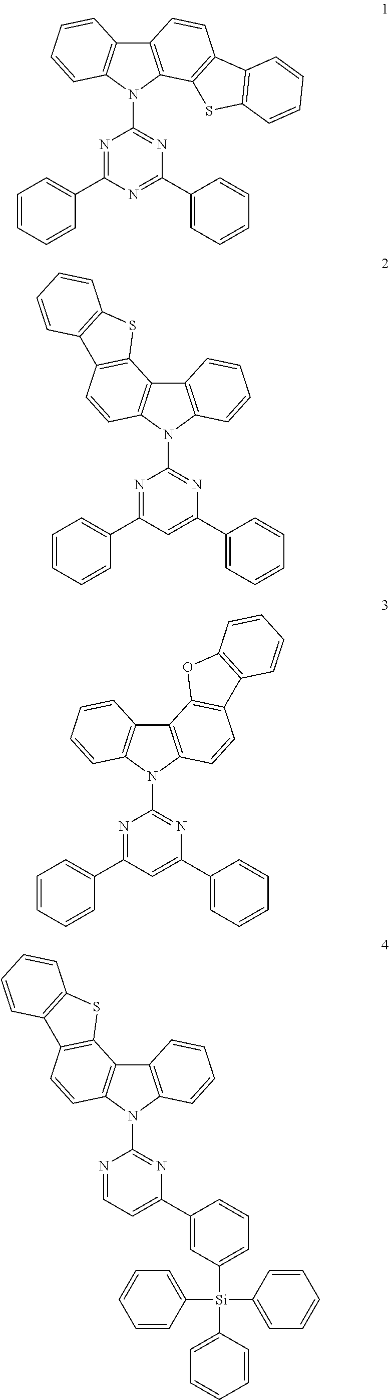

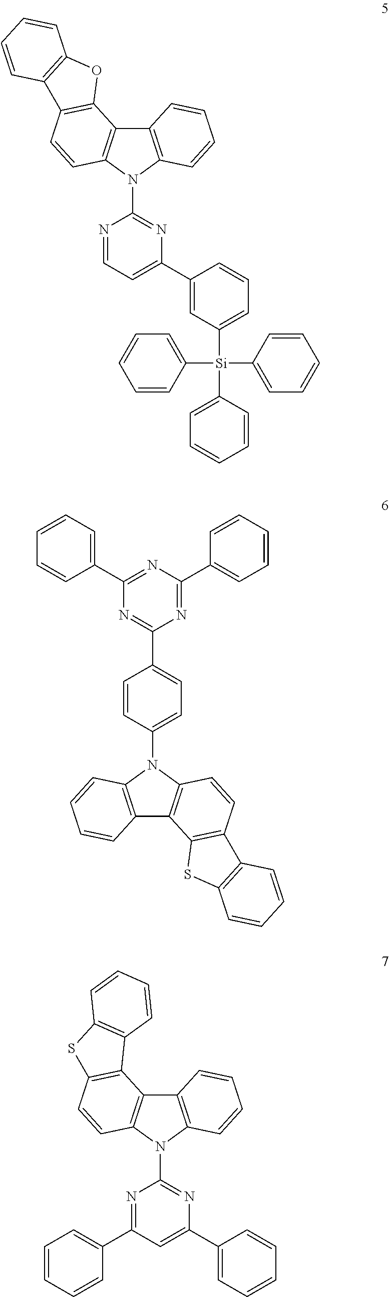 [SMILES: c1ccc([Si](c2ccccc2)(c2ccccc2)c2cccc(-c3ccnc(-n4c5ccccc5c5c6oc7ccccc7c6ccc54)n3)c2)cc1 ; c1ccc(-c2nc(-c3ccccc3)nc(-c3ccc(-n4c5ccccc5c5c6sc7ccccc7c6ccc54)cc3)n2)cc1 ; c1ccc(-c2cc(-c3ccccc3)nc(-n3c4ccccc4c4c5c(ccc43)sc3ccccc35)n2)cc1]

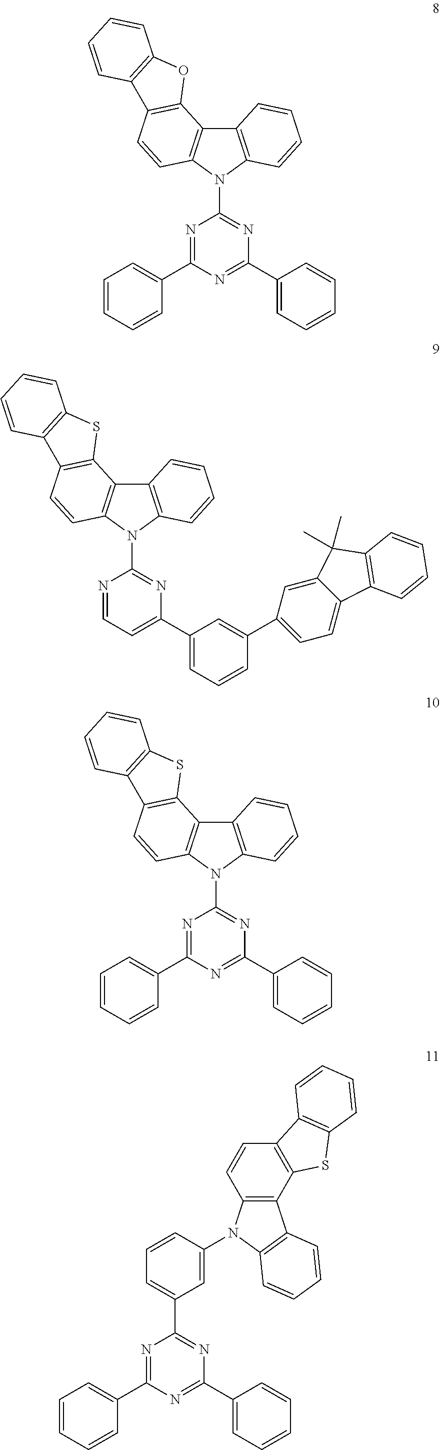

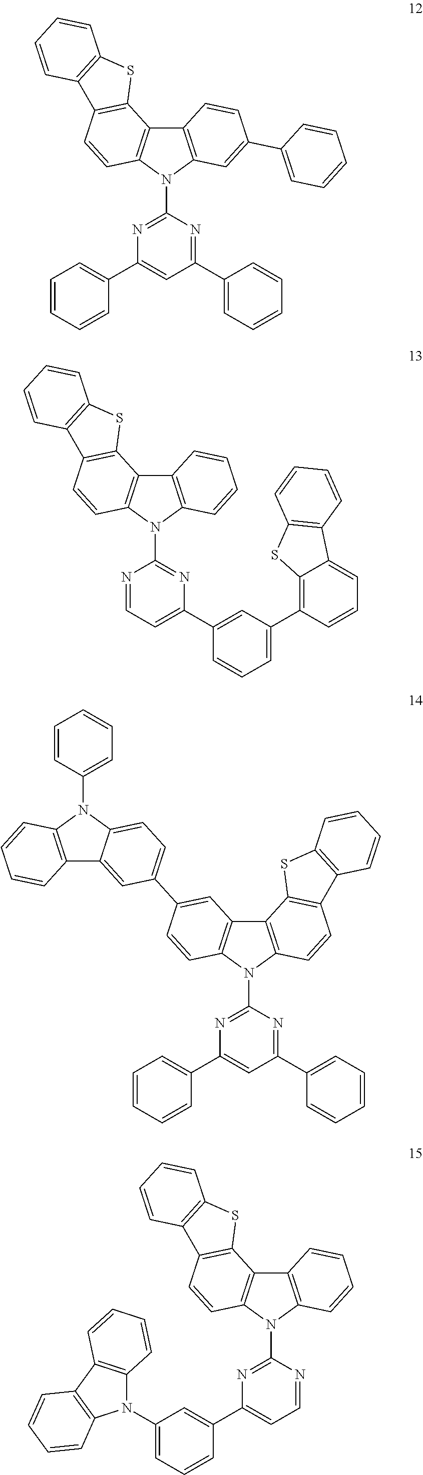

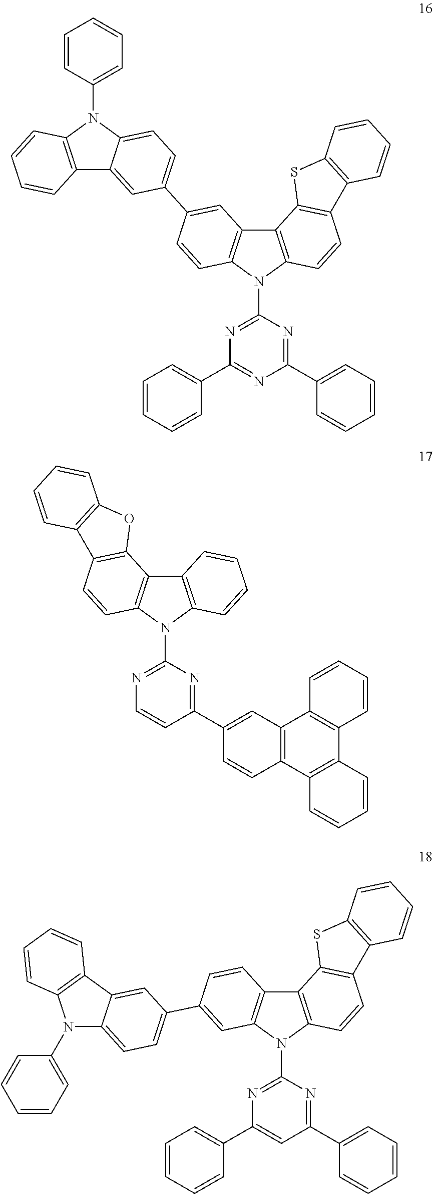 [SMILES: c1ccc(-c2nc(-c3ccccc3)nc(-n3c4ccc(-c5ccc6c(c5)c5ccccc5n6-c5ccccc5)cc4c4c5sc6ccccc6c5ccc43)n2)cc1 ; c1ccc2c(c1)oc1c2ccc2c1c1ccccc1n2-c1nccc(-c2ccc3c4ccccc4c4ccccc4c3c2)n1 ; c1ccc(-c2cc(-c3ccccc3)nc(-n3c4cc(-c5ccc6c(c5)c5ccccc5n6-c5ccccc5)ccc4c4c5sc6ccccc6c5ccc43)n2)cc1]

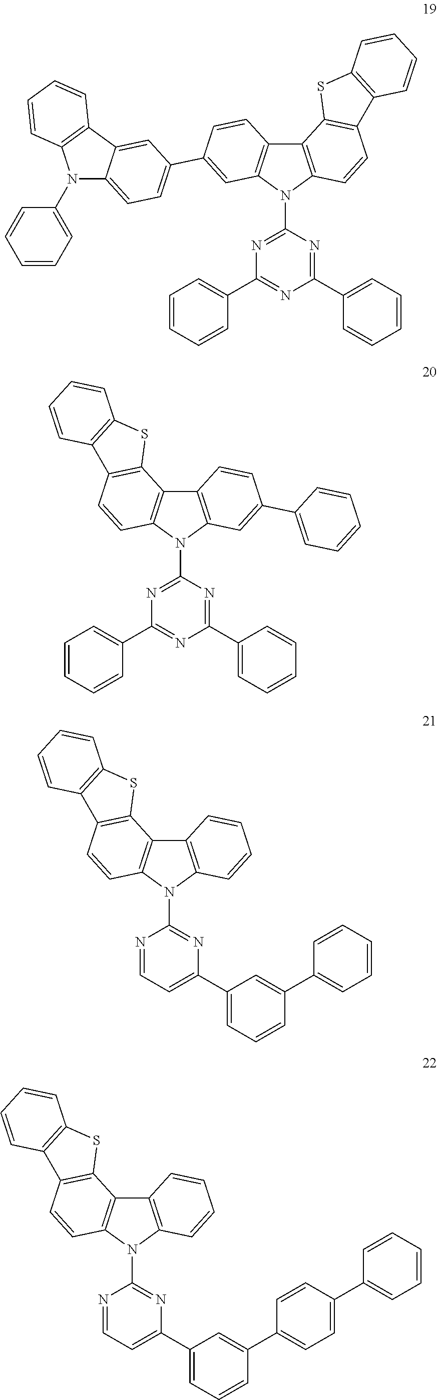

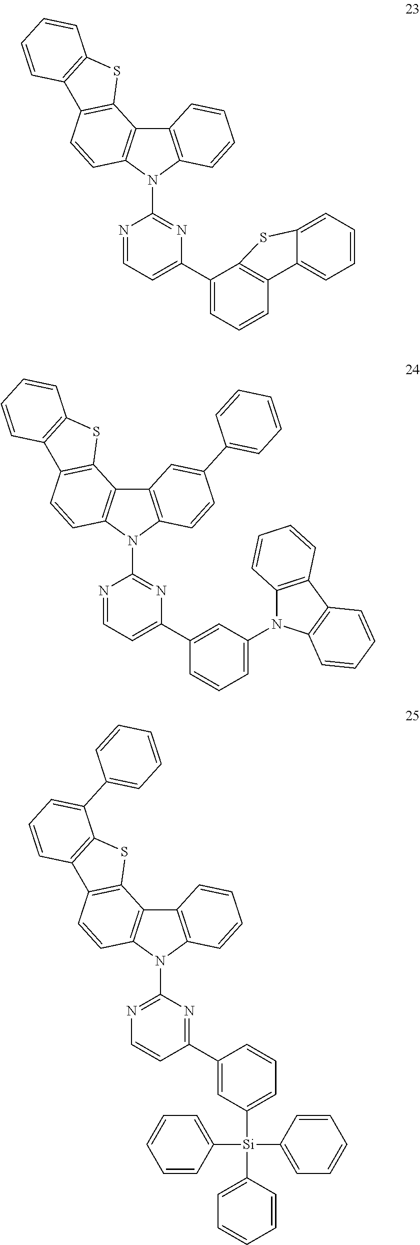 [SMILES: c1ccc2c(c1)sc1c(-c3ccnc(-n4c5ccccc5c5c6sc7ccccc7c6ccc54)n3)cccc12 ; c1ccc(-c2ccc3c(c2)c2c4sc5ccccc5c4ccc2n3-c2nccc(-c3cccc(-n4c5ccccc5c5ccccc54)c3)n2)cc1 ; c1ccc(-c2cccc3c2sc2c3ccc3c2c2ccccc2n3-c2nccc(-c3cccc([Si](c4ccccc4)(c4ccccc4)c4ccccc4)c3)n2)cc1]

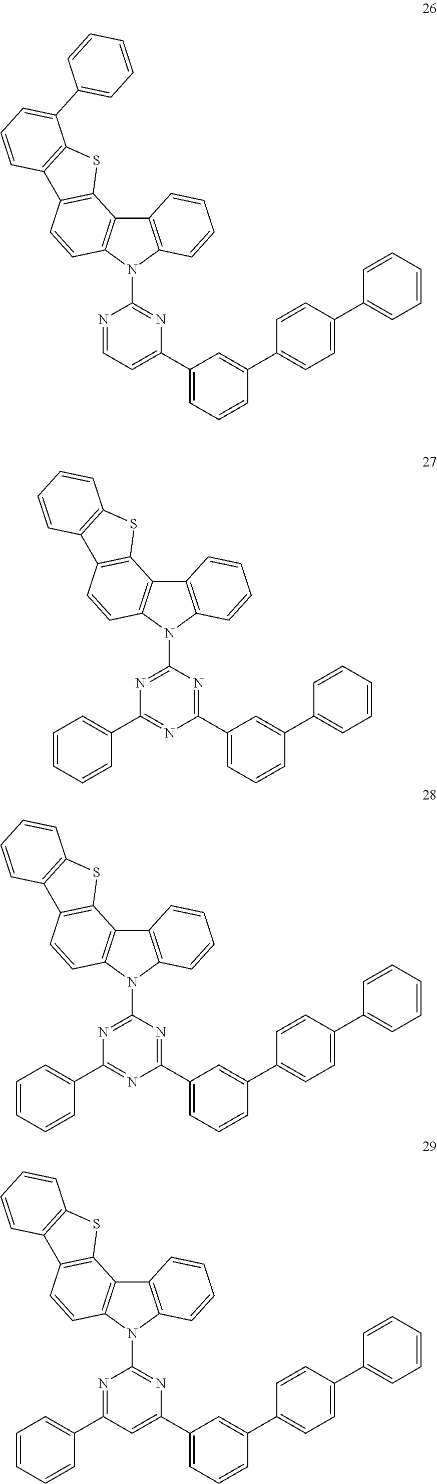

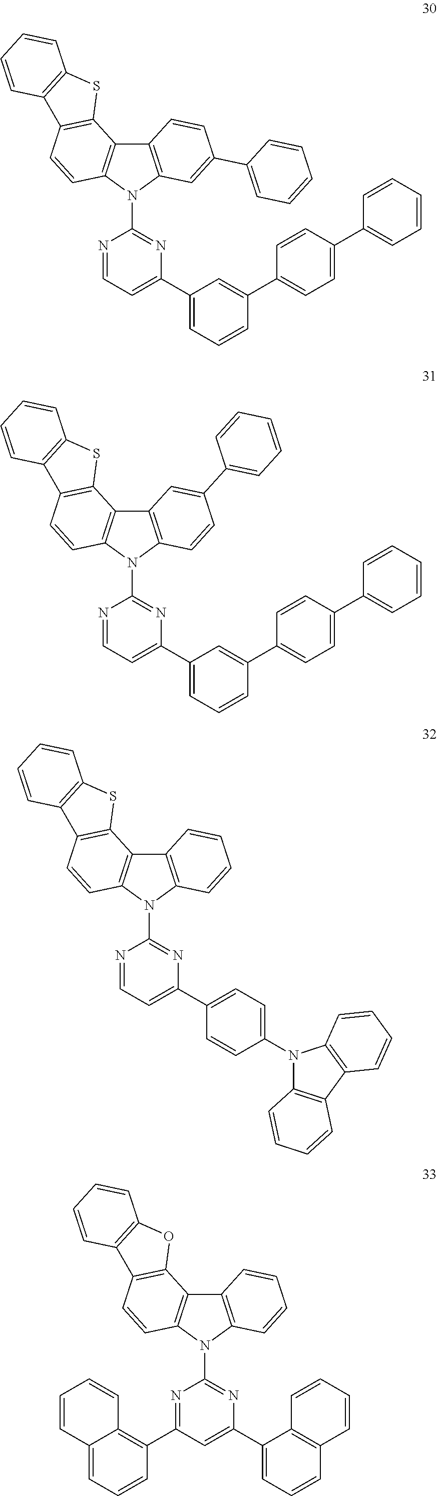 [SMILES: c1ccc(-c2ccc(-c3cccc(-c4ccnc(-n5c6cc(-c7ccccc7)ccc6c6c7sc8ccccc8c7ccc65)n4)c3)cc2)cc1 ; c1ccc(-c2ccc(-c3cccc(-c4ccnc(-n5c6ccc(-c7ccccc7)cc6c6c7sc8ccccc8c7ccc65)n4)c3)cc2)cc1 ; c1ccc2c(c1)sc1c2ccc2c1c1ccccc1n2-c1nccc(-c2ccc(-n3c4ccccc4c4ccccc43)cc2)n1 ; c1ccc2c(-c3cc(-c4cccc5ccccc45)nc(-n4c5ccccc5c5c6oc7ccccc7c6ccc54)n3)cccc2c1]

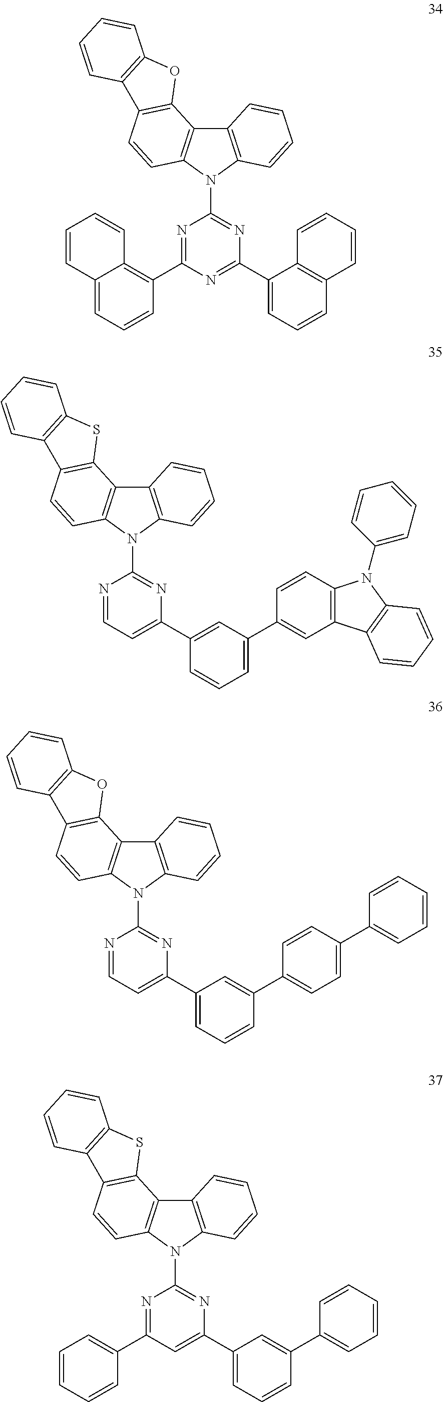 [SMILES: c1ccc2c(-c3nc(-c4cccc5ccccc45)nc(-n4c5ccccc5c5c6oc7ccccc7c6ccc54)n3)cccc2c1 ; c1ccc(-n2c3ccccc3c3cc(-c4cccc(-c5ccnc(-n6c7ccccc7c7c8sc9ccccc9c8ccc76)n5)c4)ccc32)cc1 ; c1ccc(-c2ccc(-c3cccc(-c4ccnc(-n5c6ccccc6c6c7oc8ccccc8c7ccc65)n4)c3)cc2)cc1 ; c1ccc(-c2cccc(-c3cc(-c4ccccc4)nc(-n4c5ccccc5c5c6sc7ccccc7c6ccc54)n3)c2)cc1]

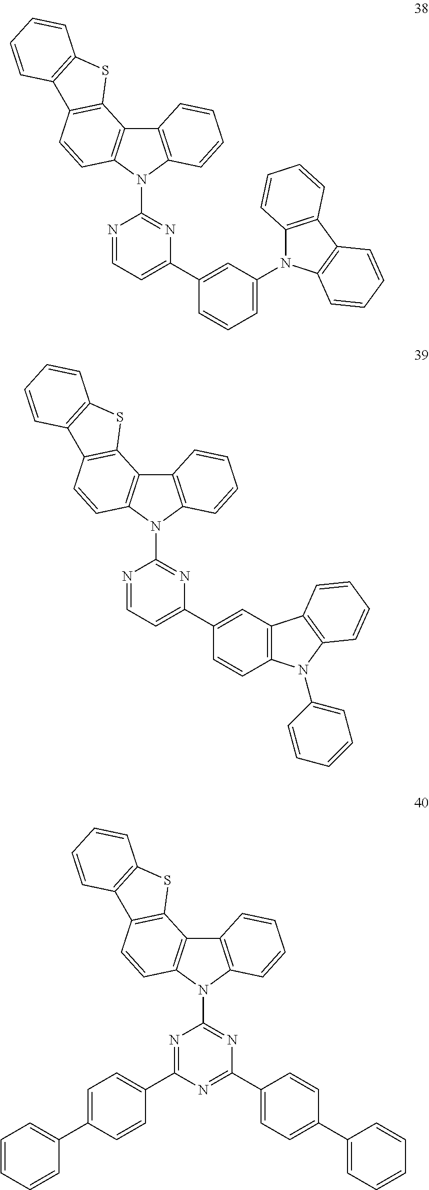 [SMILES: c1cc(-c2ccnc(-n3c4ccccc4c4c5sc6ccccc6c5ccc43)n2)cc(-n2c3ccccc3c3ccccc32)c1 ; c1ccc(-n2c3ccccc3c3cc(-c4ccnc(-n5c6ccccc6c6c7sc8ccccc8c7ccc65)n4)ccc32)cc1 ; c1ccc(-c2ccc(-c3nc(-c4ccc(-c5ccccc5)cc4)nc(-n4c5ccccc5c5c6sc7ccccc7c6ccc54)n3)cc2)cc1]

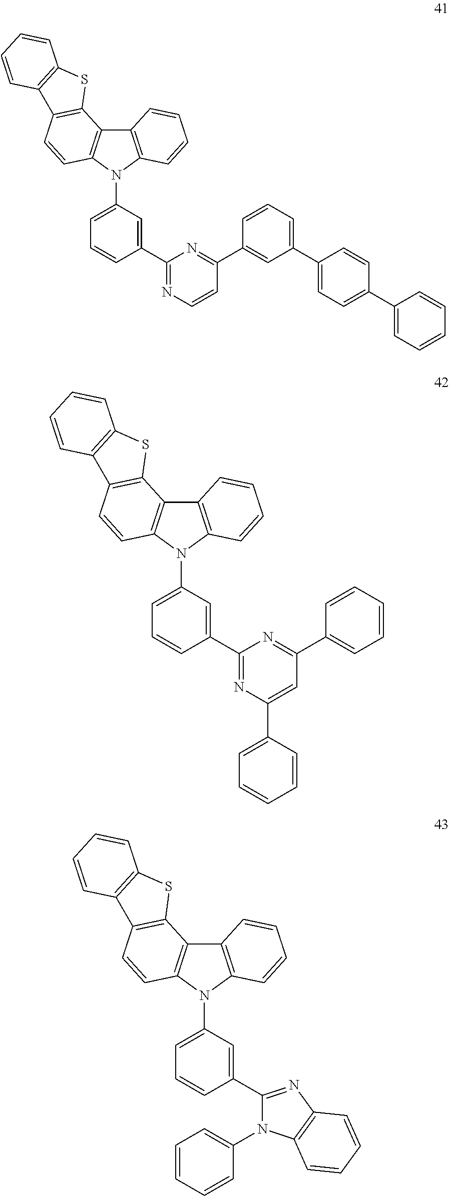 [SMILES: c1ccc(-c2ccc(-c3cccc(-c4ccnc(-c5cccc(-n6c7ccccc7c7c8sc9ccccc9c8ccc76)c5)n4)c3)cc2)cc1 ; c1ccc(-c2cc(-c3ccccc3)nc(-c3cccc(-n4c5ccccc5c5c6sc7ccccc7c6ccc54)c3)n2)cc1 ; c1ccc(-n2c(-c3cccc(-n4c5ccccc5c5c6sc7ccccc7c6ccc54)c3)nc3ccccc32)cc1]

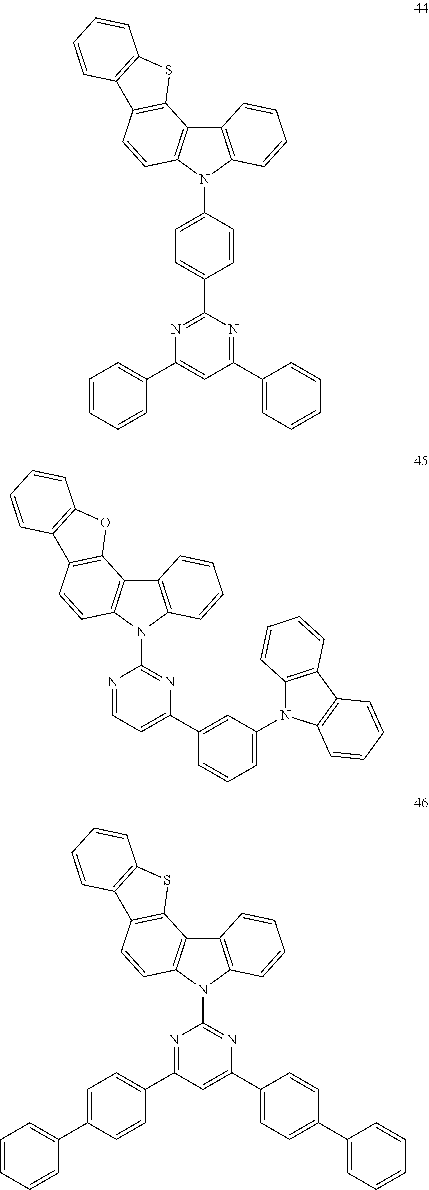 [SMILES: c1ccc(-c2cc(-c3ccccc3)nc(-c3ccc(-n4c5ccccc5c5c6sc7ccccc7c6ccc54)cc3)n2)cc1 ; c1cc(-c2ccnc(-n3c4ccccc4c4c5oc6ccccc6c5ccc43)n2)cc(-n2c3ccccc3c3ccccc32)c1 ; c1ccc(-c2ccc(-c3cc(-c4ccc(-c5ccccc5)cc4)nc(-n4c5ccccc5c5c6sc7ccccc7c6ccc54)n3)cc2)cc1]

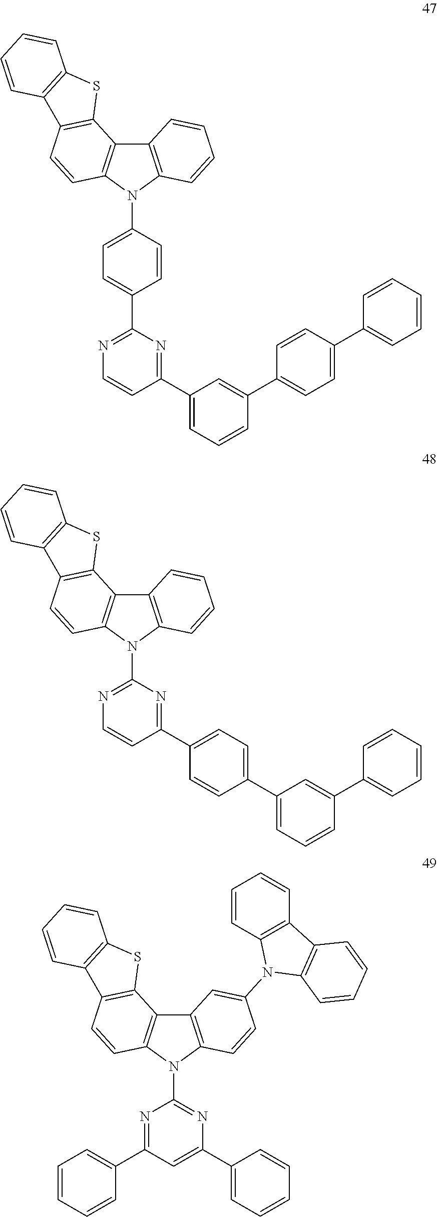

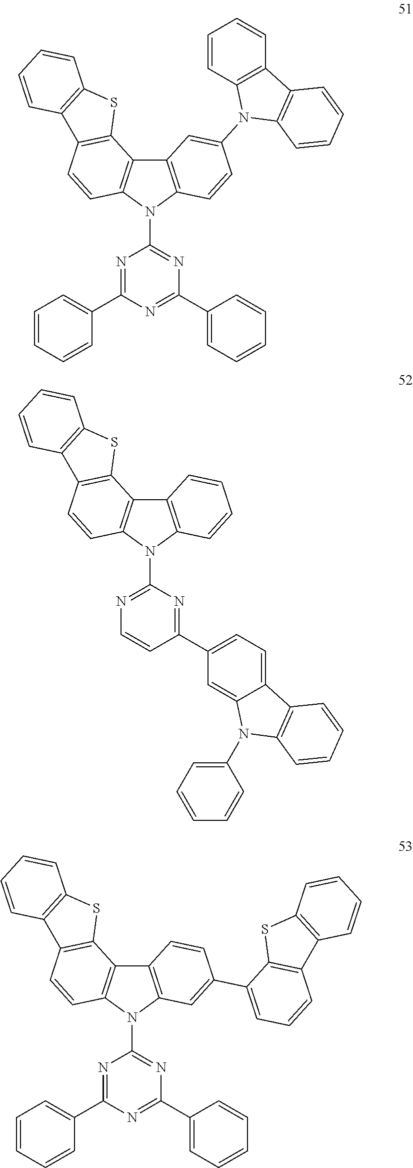

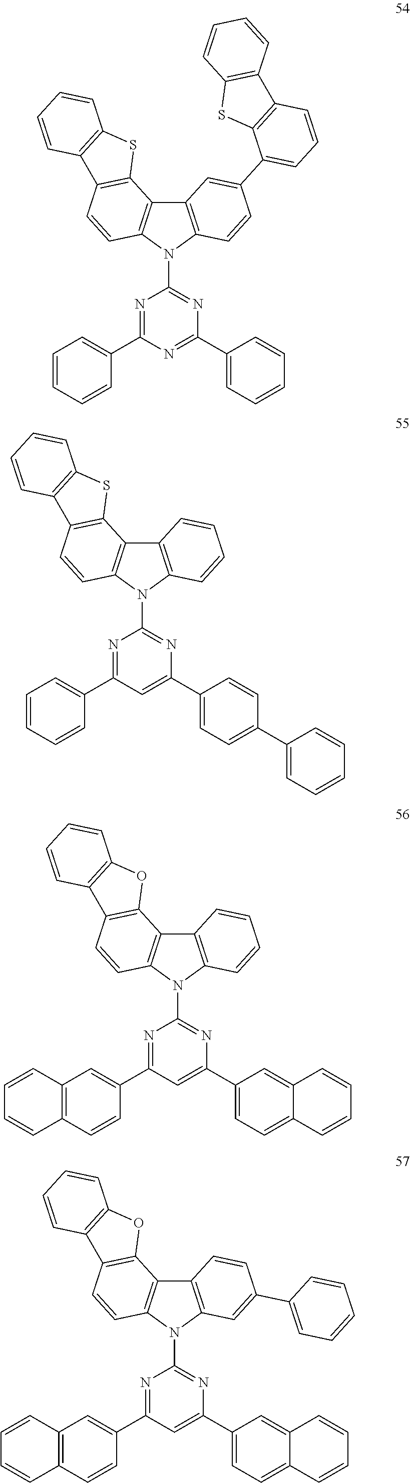

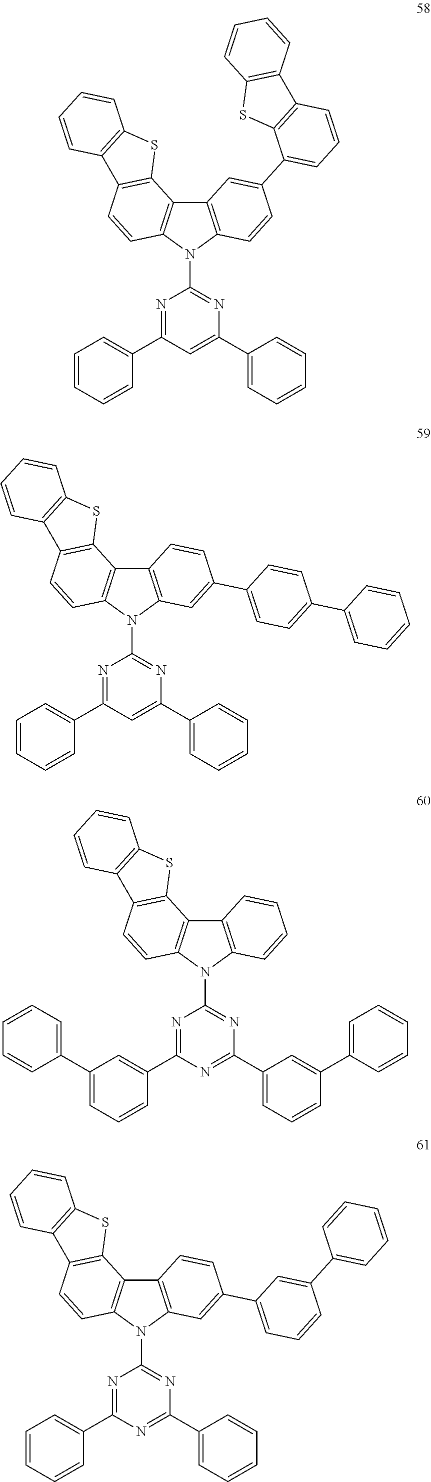 [SMILES: c1ccc(-c2cc(-c3ccccc3)nc(-n3c4ccc(-c5cccc6c5sc5ccccc56)cc4c4c5sc6ccccc6c5ccc43)n2)cc1 ; c1ccc(-c2ccc(-c3ccc4c5c6sc7ccccc7c6ccc5n(-c5nc(-c6ccccc6)cc(-c6ccccc6)n5)c4c3)cc2)cc1 ; c1ccc(-c2cccc(-c3nc(-c4cccc(-c5ccccc5)c4)nc(-n4c5ccccc5c5c6sc7ccccc7c6ccc54)n3)c2)cc1 ; c1ccc(-c2cccc(-c3ccc4c5c6sc7ccccc7c6ccc5n(-c5nc(-c6ccccc6)nc(-c6ccccc6)n5)c4c3)c2)cc1]

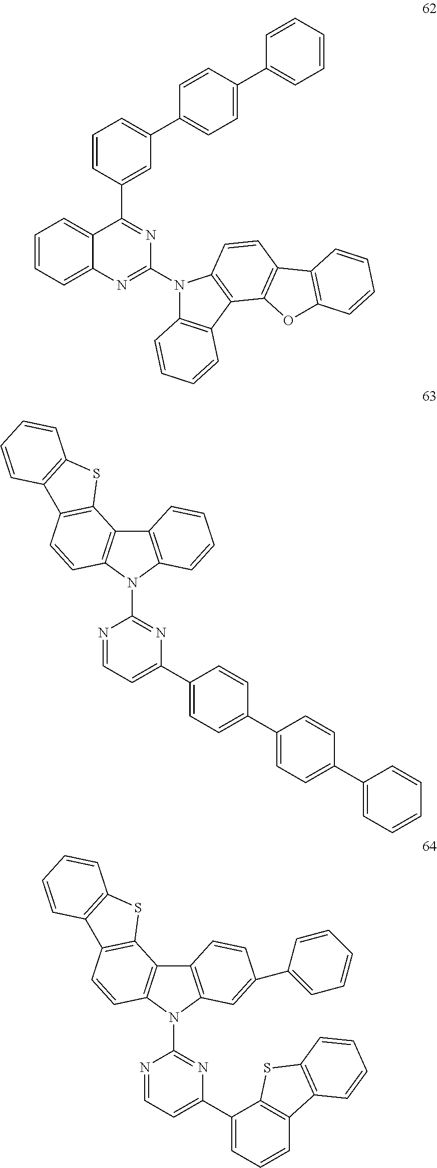 [SMILES: c1ccc(-c2ccc(-c3cccc(-c4nc(-n5c6ccccc6c6c7oc8ccccc8c7ccc65)nc5ccccc45)c3)cc2)cc1 ; c1ccc(-c2ccc(-c3ccc(-c4ccnc(-n5c6ccccc6c6c7sc8ccccc8c7ccc65)n4)cc3)cc2)cc1 ; c1ccc(-c2ccc3c4c5sc6ccccc6c5ccc4n(-c4nccc(-c5cccc6c5sc5ccccc56)n4)c3c2)cc1]

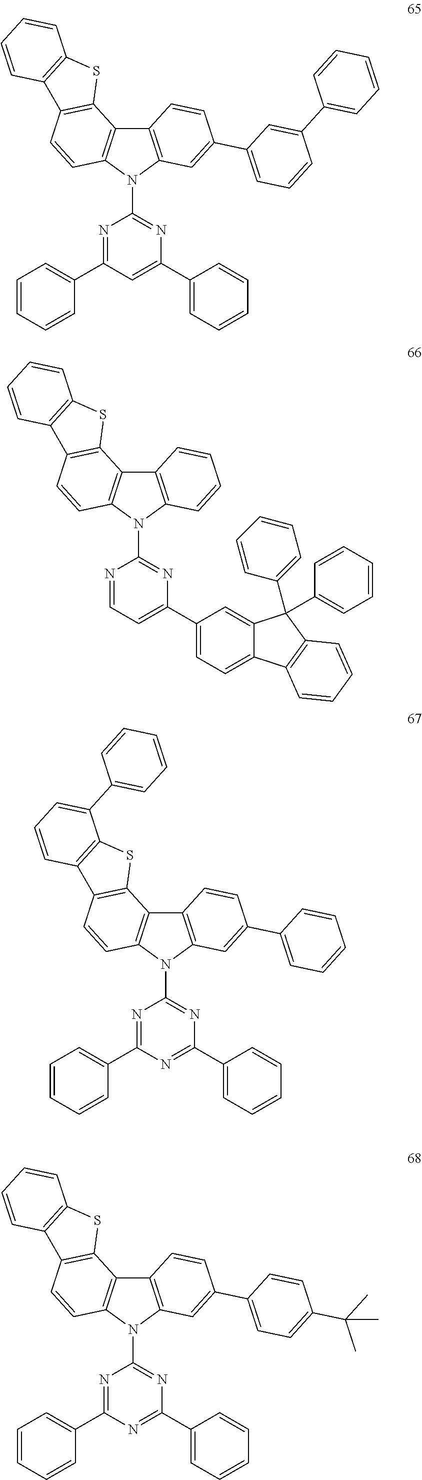

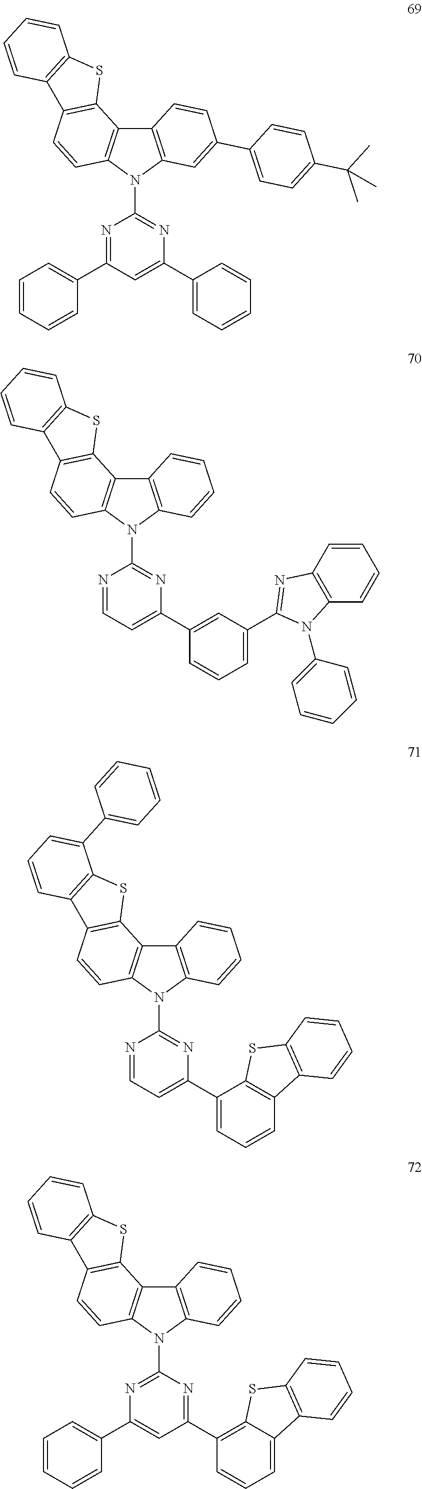

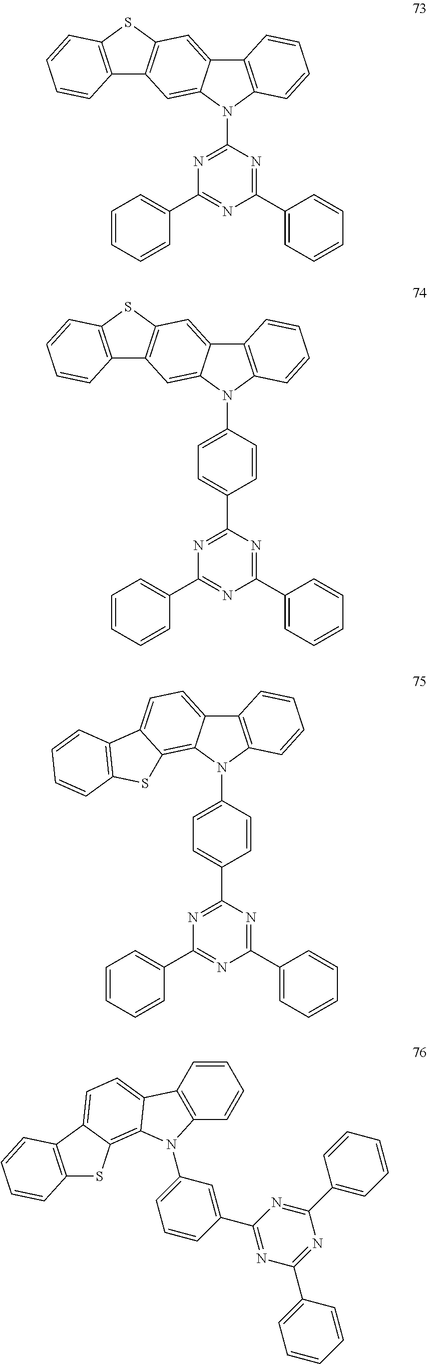 [SMILES: c1ccc(-c2nc(-c3ccccc3)nc(-n3c4ccccc4c4cc5sc6ccccc6c5cc43)n2)cc1 ; c1ccc(-c2nc(-c3ccccc3)nc(-c3ccc(-n4c5ccccc5c5cc6sc7ccccc7c6cc54)cc3)n2)cc1 ; c1ccc(-c2nc(-c3ccccc3)nc(-c3ccc(-n4c5ccccc5c5ccc6c7ccccc7sc6c54)cc3)n2)cc1 ; c1ccc(-c2nc(-c3ccccc3)nc(-c3cccc(-n4c5ccccc5c5ccc6c7ccccc7sc6c54)c3)n2)cc1]

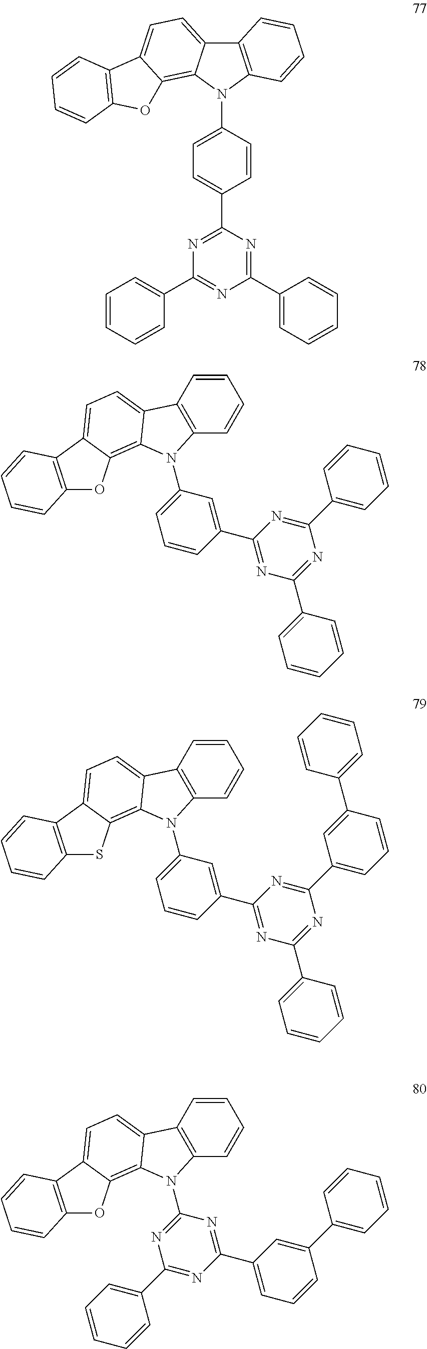

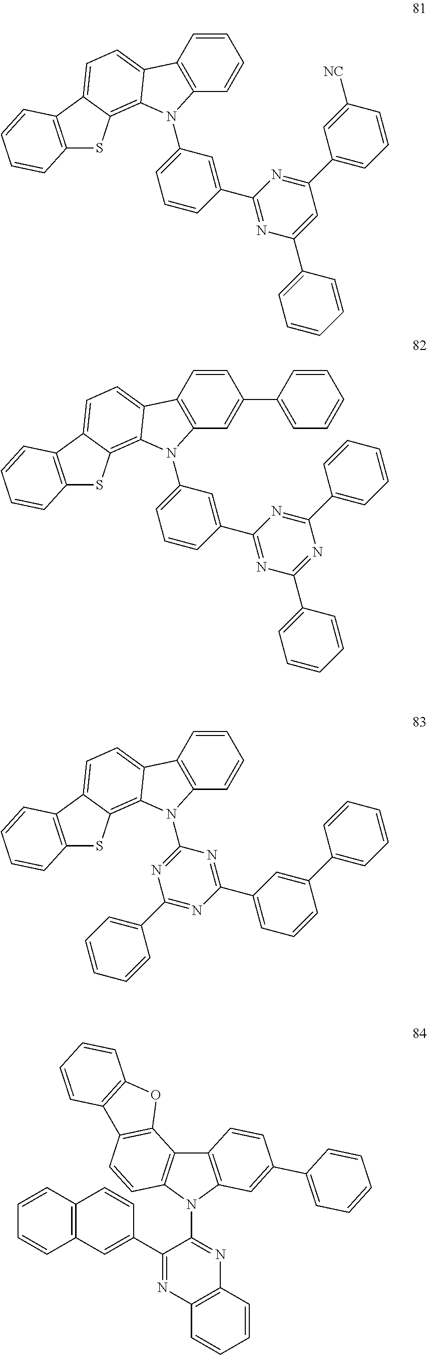

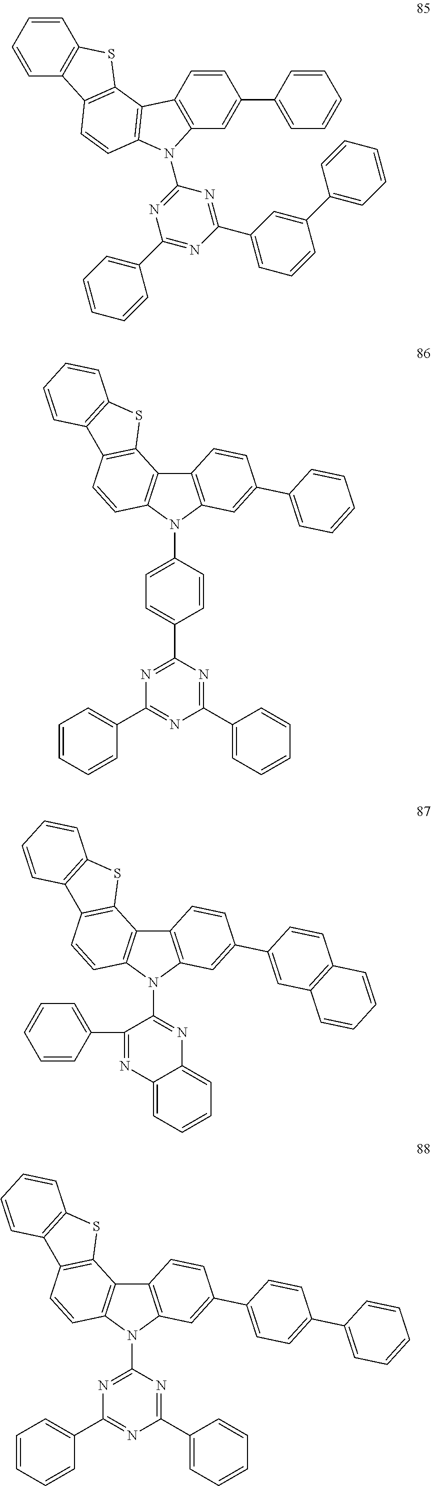 [SMILES: c1ccc(-c2cccc(-c3nc(-c4ccccc4)nc(-n4c5cc(-c6ccccc6)ccc5c5c6sc7ccccc7c6ccc54)n3)c2)cc1 ; c1ccc(-c2ccc3c4c5sc6ccccc6c5ccc4n(-c4ccc(-c5nc(-c6ccccc6)nc(-c6ccccc6)n5)cc4)c3c2)cc1 ; c1ccc(-c2nc3ccccc3nc2-n2c3cc(-c4ccc5ccccc5c4)ccc3c3c4sc5ccccc5c4ccc32)cc1 ; c1ccc(-c2ccc(-c3ccc4c5c6sc7ccccc7c6ccc5n(-c5nc(-c6ccccc6)nc(-c6ccccc6)n5)c4c3)cc2)cc1]

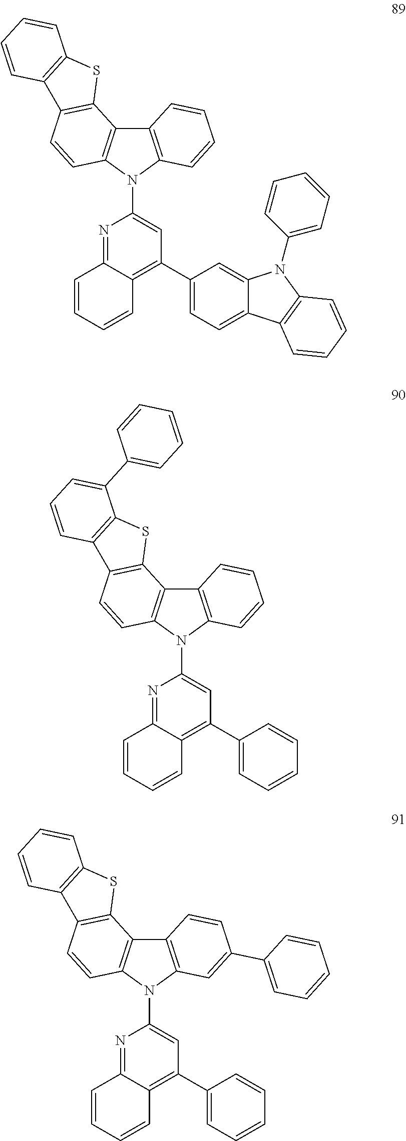 [SMILES: c1ccc(-n2c3ccccc3c3ccc(-c4cc(-n5c6ccccc6c6c7sc8ccccc8c7ccc65)nc5ccccc45)cc32)cc1 ; c1ccc(-c2cc(-n3c4ccccc4c4c5sc6c(-c7ccccc7)cccc6c5ccc43)nc3ccccc23)cc1 ; c1ccc(-c2ccc3c4c5sc6ccccc6c5ccc4n(-c4cc(-c5ccccc5)c5ccccc5n4)c3c2)cc1]

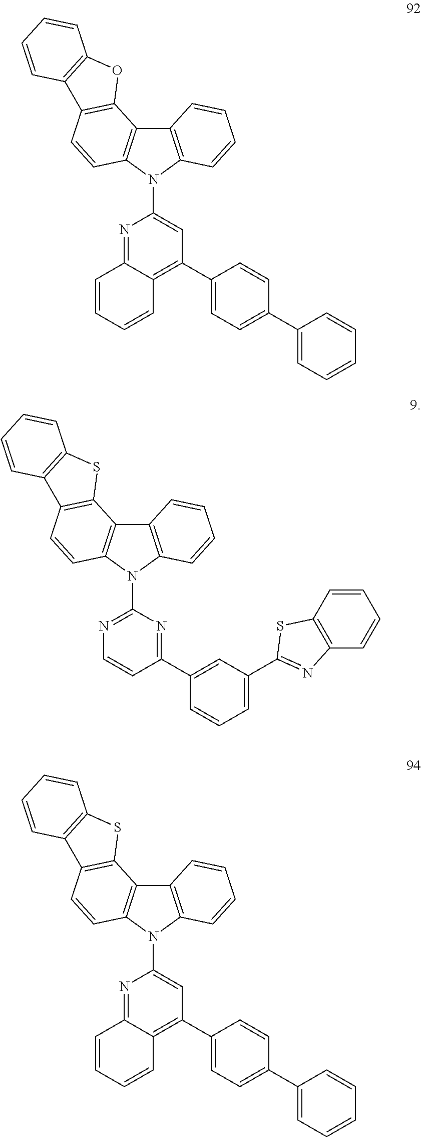 [SMILES: c1ccc(-c2ccc(-c3cc(-n4c5ccccc5c5c6oc7ccccc7c6ccc54)nc4ccccc34)cc2)cc1 ; c1cc(-c2ccnc(-n3c4ccccc4c4c5sc6ccccc6c5ccc43)n2)cc(-c2nc3ccccc3s2)c1 ; c1ccc(-c2ccc(-c3cc(-n4c5ccccc5c5c6sc7ccccc7c6ccc54)nc4ccccc34)cc2)cc1]

8. An organic electroluminescent device comprising the electron transport material as defined in claim 1.

9. The organic electroluminescent device according to claim 8, further comprising a reducing dopant.

10. The organic electroluminescent device according to claim 9, wherein the reducing dopant is one or more selected from the group consisting of an alkaline metal, an alkaline earth metal, a rare-earth metal, an oxide of an alkaline metal, a halide of an alkaline metal, an oxide of an alkaline earth metal, a halide of an alkaline earth metal, an oxide of a rare-earth metal, a halide of a rare-earth metal, an organic complex of an alkaline metal, an organic complex of an alkaline earth metal, and an organic complex of a rare-earth metal.
